(19) United States
(12) Patent Application Publication
(10) Pub. No.: US 2026/0052835 A1
(43) Pub. Date: Feb. 19, 2026

(54) HOLE TRANSPORT MATERIAL, ORGANIC ELECTROLUMINESCENT DEVICE, FORMULATION, DISPLAY APPARATUS AND LIGHTING APPARATUS

(71) Applicant: Anhui Huaxian New Material Technology Co., Ltd., Fuyang (CN)

(72) Inventors: Zhunmo SUN, Fuyang (CN); Lei ZHANG, Fuyang (CN); Yunqi LIU, Fuyang (CN); Yangbo WU, Fuyang (CN); Nate AMITE, Fuyang (CN); Yueyang LUO, Fuyang (CN); Minyu RAO, Fuyang (CN); Xiaoyu ZHAO, Fuyang (CN)

(73) Assignee: Anhui Huaxian New Material Technology Co., Ltd., Fuyang (CN)

(21) Appl. No.: 19/295,296

(22) Filed: Aug. 8, 2025

(30) Foreign Application Priority Data
Aug. 14, 2024 (CN) 202411112059.X
Mar. 24, 2025 (WO) PCT/CN2025/084275

(51) Int. Cl. H10K 50/15; C09K 11/06; H10K 50/11; H10K 85/60

(52) U.S. Cl. CPC H10K 50/15; C09K 11/06; H10K 50/11; H10K 85/626; H10K 85/633; H10K 85/636; H10K 85/654; H10K 85/656; H10K 85/6572; H10K 85/6574; H10K 85/658; C09K 2211/1029; C09K 2211/1088

(57) ABSTRACT

A hole transport material uses triarylamine as a parent nucleus, and uses an aryl-substituted, alkyl-substituted or aryl and alkyl co-substituted fluorenyl group, an aryl-substituted or alkyl-substituted fluorenyl group or a heteroaryl group, and a ortho-substituted aryl group as three side chains to combine with each other, which enables a compound to maintain a high stability. In addition, when being used to fabricate an organic electroluminescent (EL) element, the hole transport material effectively improves a light-emitting efficiency and a working life of the organic EL device, such that the hole transport material has a good application prospect.

### CROSS-REFERENCE TO RELATED APPLICATIONS

This application claims priority to Chinese Patent Application No. 202411112059.X, filed on Aug. 14, 2024, titled “HOLE TRANSPORT MATERIAL, ORGANIC LIGHT-EMITTING DEVICE AND DISPLAY OR LIGHTING APPARATUS”, and claims priority to International Patent Application No. PCT/CN2025/084275, filed on Mar. 24, 2025, titled “HOLE TRANSPORT MATERIAL, ORGANIC LIGHT-EMITTING DEVICE AND DISPLAY OR LIGHTING APPARATUS”, which are incorporated herein by reference in their entirety.

### TECHNICAL FIELD

The present disclosure relates to the technical field of organic optoelectronic material preparation, and specifically, to a hole transport material, an organic electroluminescent device, a formulation, a display apparatus and a lighting apparatus.

### BACKGROUND

An organic light-emitting diode (OLED), also referred to as an organic electroluminescent device, relates to a technology in which holes and electrons are injected into a light-emitting layer respectively from an anode and a cathode by applying a voltage to the electroluminescent device, and the injected holes combine with the injected electrons to form excitons for light emission, such that electrical energy can be converted into optical energy through an organic optoelectronic material.

Generally, an organic optoelectronic material layer has a multilayer structure composed of different materials to improve a efficiency and a stability of an organic light-emitting device. For example, the organic optoelectronic material layer may include a hole injection layer, a hole transport layer, a light-emitting layer, an electron transport layer, an electron injection layer, etc. At present, hole transport materials generally suffer from poor thermal stability, and low highest occupied molecular orbital (HOMO) value and triplet energy level. When the hole transport materials are applied to organic light-emitting devices, there is still a need to improve driving voltages, light-emitting efficiencies and working lives of the devices.

Therefore, there is a need to develop organic optoelectronic materials with a good stability as well as excellent light-emitting properties, and to find suitable OLED optoelectronic functional materials for OLED devices so as to solve the above-described problems.

### SUMMARY

A purpose of embodiments of the present disclosure is to provide one or more hole transport materials for an organic electroluminescent device. In order to solve the above-described technical problems, embodiments of the present disclosure provide a triarylamine-type hole transport material obtained by combining an ortho-substituted aryl group with a substituted fluorenyl group, an organic light-emitting diode (OLED) containing the hole transport material, a composition, a formulation, a display apparatus and a lighting apparatus. The provided hole transport material uses triarylamine as a parent nucleus, and uses an aryl-substituted, alkyl-substituted or aryl and alkyl co-substituted fluorenyl group, an aryl-substituted or alkyl-substituted fluorenyl group or a heteroaryl group, and an ortho-substituted aryl group as three side chains to combine with each other, which enables the hole transport material to maintain a high stability and enables a device to have a high efficiency.

The hole transport material provided in embodiments of the present disclosure is realized by means of the following technical solution.

The hole transport material contains a compound with a structure as shown in formula (I) below:

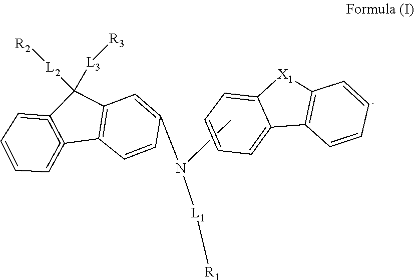

In formula (I), X₁ independently represents CRₐR_{b}, NR_{c}, an O atom or an S atom; and Rₐ, R_{b} and R_{c} are each independently selected from a C1-C24 alkyl group, a C6-C30 aryl group and a C5-C30 heteroaryl group;
L₁ is selected from a single bond and a substituted or unsubstituted C6-C30 arylene group, the substituted C6-C30 arylene group having mono-substitution, di-substitution or a maximum permissible number of substitutions, and in a case of a substituent is involved, the substituent being hydrogen, deuterium, a C1-C24 alkyl group or a monocyclic, annelated, bridged cyclic, spirocyclic or fused cyclic C3-C20 cycloalkyl group; R₁ is selected from a C1-C24 alkyl-substituted, or monocyclic, annelated, bridged cyclic, spirocyclic or fused cyclic C3-C20 cycloalkyl-substituted or unsubstituted C6-C30 aryl group, and a C6-C30 aryl-substituted or unsubstituted C4-C36 heteroaryl group;
L₂ and L₃ are each independently selected from a single bond, a C1-C24 alkyl group, a C3-C20 cycloalkyl group and a substituted or unsubstituted C6-C30 aryl group, in a case of a substituent is involved, the substituent being a C3-C6 cycloalkyl group; and R₂ and R₃ are each independently selected from a C1-C10 alkyl group and a C6-C30 aryl group. The compound as shown in formula (I) may be partially or fully deuterated.

In some embodiments, in formula (I), L₂ and L₃ are each independently selected from a single bond, an n-propyl group, an isopropyl group, a C3-C6 cycloalkyl group and a C1-C6 alkyl-substituted phenyl group; and R₂ and R³ are each independently selected from a methyl group and a phenyl group.

In some embodiments, the structure shown in formula (I) may be any one of structures shown in formulas (I-1) to (I-2):

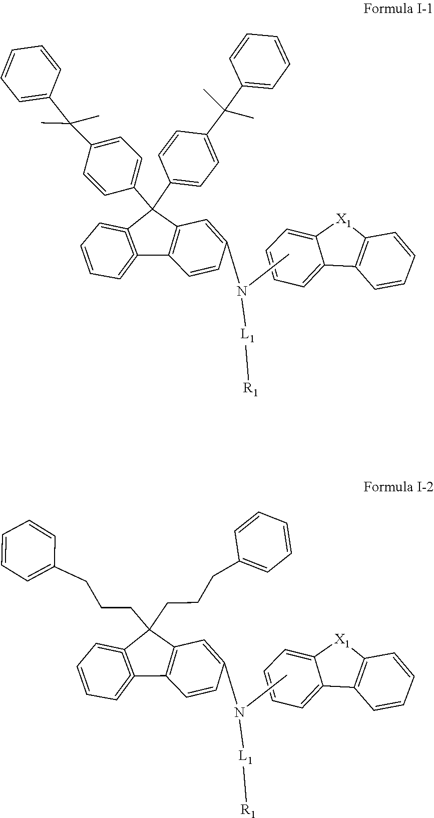

Here, X₁, L₁ and R₁ are as defined in the embodiments above.

In some embodiments, in a case where X₁ represents CRₐR_{b}, Rₐ and R_{b} are each independently selected from a methyl group and a phenyl group, or Rₐ and R_{b} is connected to each other to form a C3-C10 cycloalkyl group. In a case where X₁ represents NR_{c}, R_{c} is a phenyl group.

In some embodiments, in formula (I), L₁ is selected from a single bond and a C1-C10 alkyl-substituted C6-C30 arylenen group; and R₁ is independently selected from a C1-C5 alkyl-substituted, C3-C10 cycloalkyl-substituted or unsubstituted C6-C30 aryl group, and a C6-C30 aryl-substituted or unsubstituted C4-C36 heteroaryl group.

In some embodiments, L₁ is independently selected from a single bond, a phenyl group, a biphenyl group, a methylphenyl group, a dimethylphenyl group and an adamantyl-substituted phenyl group; and R₁ is independently selected from a phenyl group, a methylphenyl group, a dimethylphenyl group, a phenylcarbazolyl group, a dibenzofuranyl group and an adamantyl group.

One or more embodiments of the present disclosure provide a hole transport material having any chemical structure selected from chemical structures shown below:

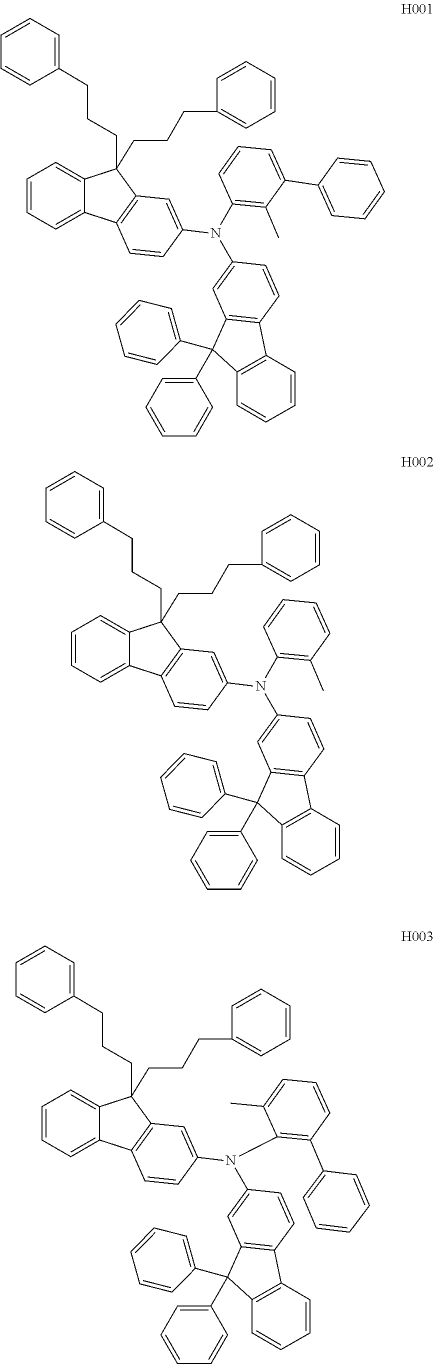 [SMILES: Cc1c(-c2ccccc2)cccc1N(c1ccc2c(c1)C(CCCc1ccccc1)(CCCc1ccccc1)c1ccccc1-2)c1ccc2c(c1)C(c1ccccc1)(c1ccccc1)c1ccccc1-2 ; Cc1ccccc1N(c1ccc2c(c1)C(CCCc1ccccc1)(CCCc1ccccc1)c1ccccc1-2)c1ccc2c(c1)C(c1ccccc1)(c1ccccc1)c1ccccc1-2 ; Cc1cccc(-c2ccccc2)c1N(c1ccc2c(c1)C(CCCc1ccccc1)(CCCc1ccccc1)c1ccccc1-2)c1ccc2c(c1)C(c1ccccc1)(c1ccccc1)c1ccccc1-2]

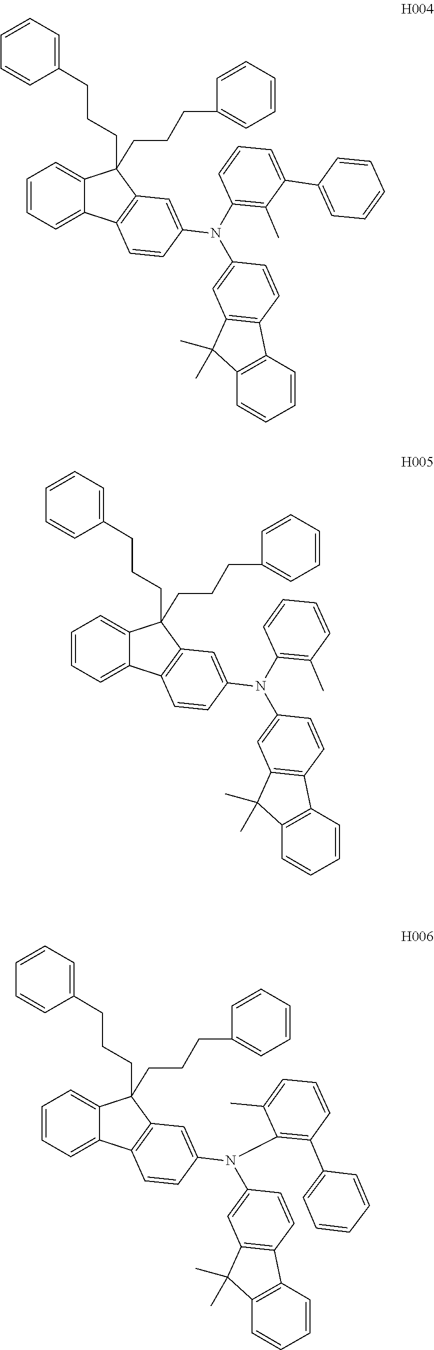 [SMILES: Cc1c(-c2ccccc2)cccc1N(c1ccc2c(c1)C(C)(C)c1ccccc1-2)c1ccc2c(c1)C(CCCc1ccccc1)(CCCc1ccccc1)c1ccccc1-2 ; Cc1ccccc1N(c1ccc2c(c1)C(C)(C)c1ccccc1-2)c1ccc2c(c1)C(CCCc1ccccc1)(CCCc1ccccc1)c1ccccc1-2 ; Cc1cccc(-c2ccccc2)c1N(c1ccc2c(c1)C(C)(C)c1ccccc1-2)c1ccc2c(c1)C(CCCc1ccccc1)(CCCc1ccccc1)c1ccccc1-2]

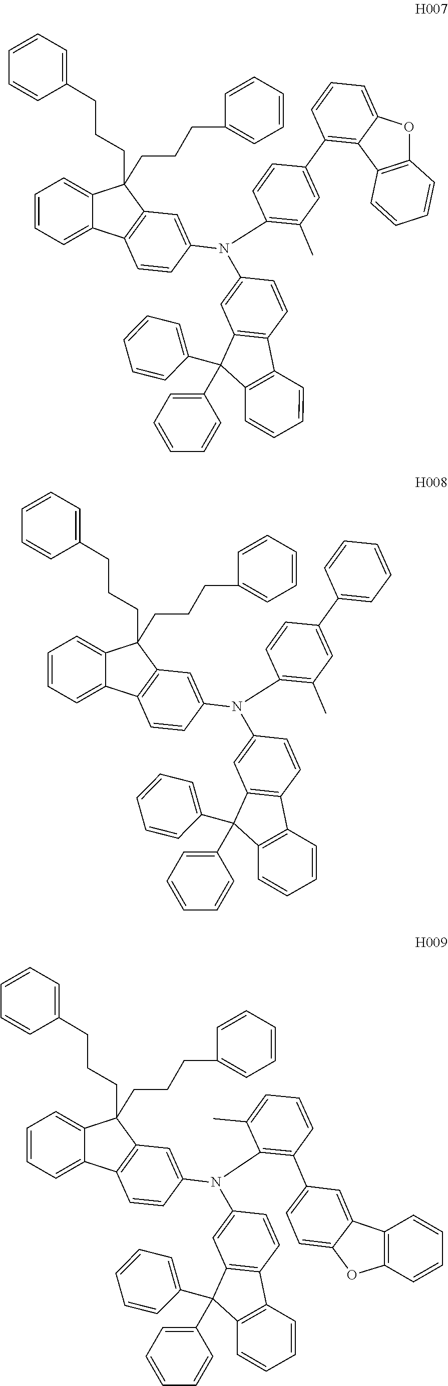 [SMILES: Cc1cc(-c2cccc3oc4ccccc4c23)ccc1N(c1ccc2c(c1)C(CCCc1ccccc1)(CCCc1ccccc1)c1ccccc1-2)c1ccc2c(c1)C(c1ccccc1)(c1ccccc1)c1ccccc1-2 ; Cc1cc(-c2ccccc2)ccc1N(c1ccc2c(c1)C(CCCc1ccccc1)(CCCc1ccccc1)c1ccccc1-2)c1ccc2c(c1)C(c1ccccc1)(c1ccccc1)c1ccccc1-2 ; Cc1cccc(-c2ccc3oc4ccccc4c3c2)c1N(c1ccc2c(c1)C(CCCc1ccccc1)(CCCc1ccccc1)c1ccccc1-2)c1ccc2c(c1)C(c1ccccc1)(c1ccccc1)c1ccccc1-2]

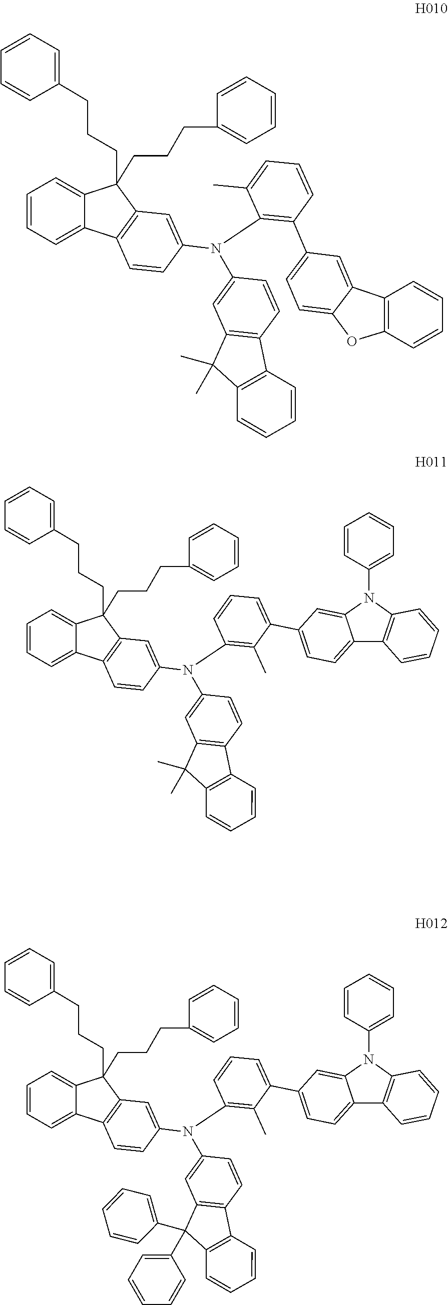 [SMILES: Cc1cccc(-c2ccc3oc4ccccc4c3c2)c1N(c1ccc2c(c1)C(C)(C)c1ccccc1-2)c1ccc2c(c1)C(CCCc1ccccc1)(CCCc1ccccc1)c1ccccc1-2 ; Cc1c(-c2ccc3c4ccccc4n(-c4ccccc4)c3c2)cccc1N(c1ccc2c(c1)C(C)(C)c1ccccc1-2)c1ccc2c(c1)C(CCCc1ccccc1)(CCCc1ccccc1)c1ccccc1-2 ; Cc1c(-c2ccc3c4ccccc4n(-c4ccccc4)c3c2)cccc1N(c1ccc2c(c1)C(CCCc1ccccc1)(CCCc1ccccc1)c1ccccc1-2)c1ccc2c(c1)C(c1ccccc1)(c1ccccc1)c1ccccc1-2]

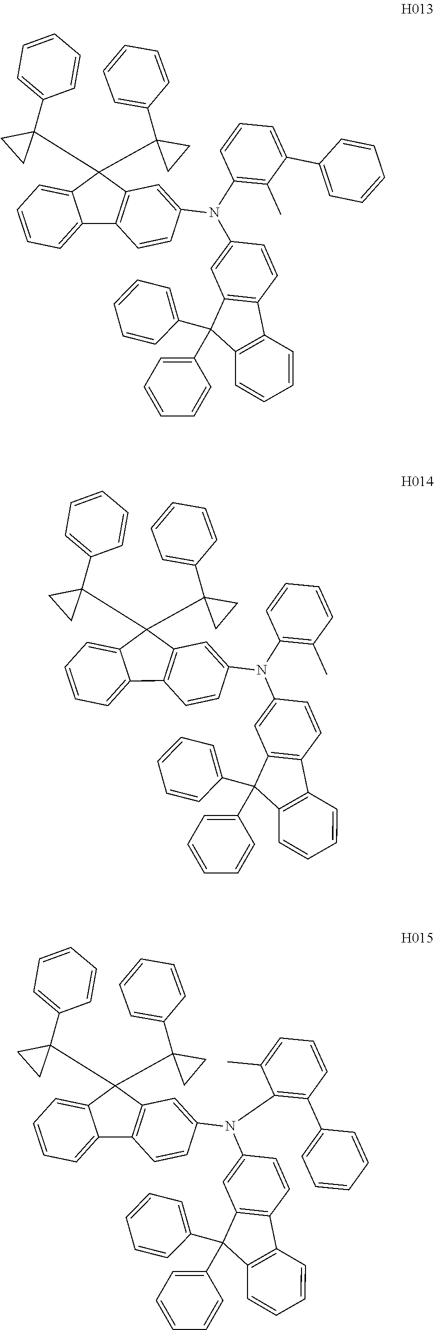 [SMILES: Cc1c(-c2ccccc2)cccc1N(c1ccc2c(c1)C(c1ccccc1)(c1ccccc1)c1ccccc1-2)c1ccc2c(c1)C(C1(c3ccccc3)CC1)(C1(c3ccccc3)CC1)c1ccccc1-2 ; Cc1ccccc1N(c1ccc2c(c1)C(c1ccccc1)(c1ccccc1)c1ccccc1-2)c1ccc2c(c1)C(C1(c3ccccc3)CC1)(C1(c3ccccc3)CC1)c1ccccc1-2 ; Cc1cccc(-c2ccccc2)c1N(c1ccc2c(c1)C(c1ccccc1)(c1ccccc1)c1ccccc1-2)c1ccc2c(c1)C(C1(c3ccccc3)CC1)(C1(c3ccccc3)CC1)c1ccccc1-2]

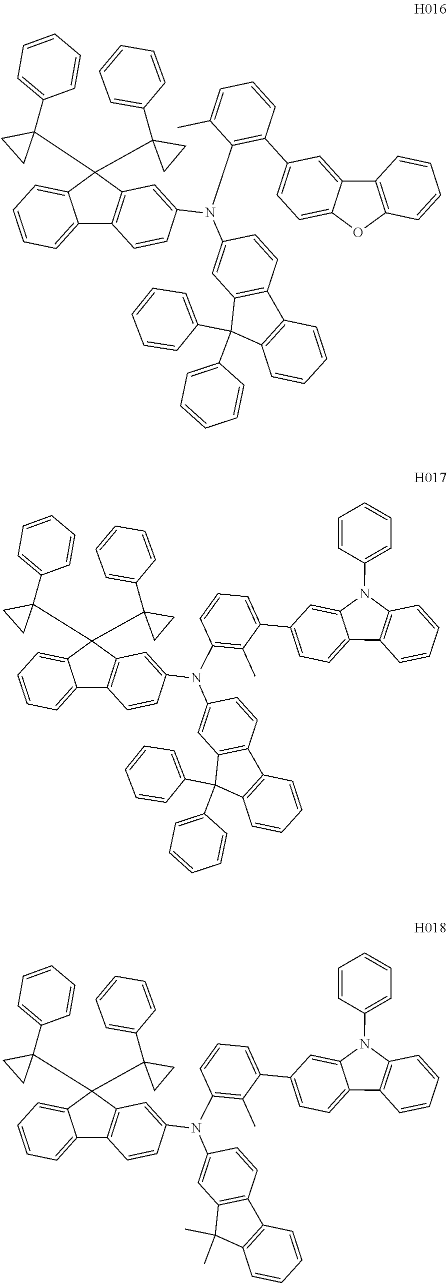 [SMILES: Cc1cccc(-c2ccc3oc4ccccc4c3c2)c1N(c1ccc2c(c1)C(c1ccccc1)(c1ccccc1)c1ccccc1-2)c1ccc2c(c1)C(C1(c3ccccc3)CC1)(C1(c3ccccc3)CC1)c1ccccc1-2 ; Cc1c(-c2ccc3c4ccccc4n(-c4ccccc4)c3c2)cccc1N(c1ccc2c(c1)C(c1ccccc1)(c1ccccc1)c1ccccc1-2)c1ccc2c(c1)C(C1(c3ccccc3)CC1)(C1(c3ccccc3)CC1)c1ccccc1-2 ; Cc1c(-c2ccc3c4ccccc4n(-c4ccccc4)c3c2)cccc1N(c1ccc2c(c1)C(C)(C)c1ccccc1-2)c1ccc2c(c1)C(C1(c3ccccc3)CC1)(C1(c3ccccc3)CC1)c1ccccc1-2]

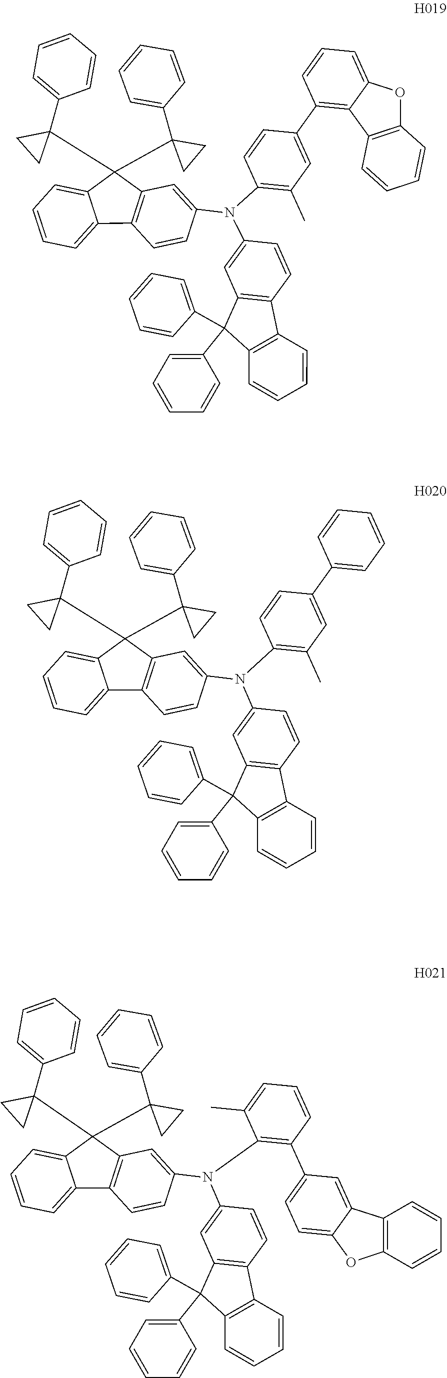 [SMILES: Cc1cc(-c2cccc3oc4ccccc4c23)ccc1N(c1ccc2c(c1)C(c1ccccc1)(c1ccccc1)c1ccccc1-2)c1ccc2c(c1)C(C1(c3ccccc3)CC1)(C1(c3ccccc3)CC1)c1ccccc1-2 ; Cc1cc(-c2ccccc2)ccc1N(c1ccc2c(c1)C(c1ccccc1)(c1ccccc1)c1ccccc1-2)c1ccc2c(c1)C(C1(c3ccccc3)CC1)(C1(c3ccccc3)CC1)c1ccccc1-2 ; Cc1cccc(-c2ccc3oc4ccccc4c3c2)c1N(c1ccc2c(c1)C(c1ccccc1)(c1ccccc1)c1ccccc1-2)c1ccc2c(c1)C(C1(c3ccccc3)CC1)(C1(c3ccccc3)CC1)c1ccccc1-2]

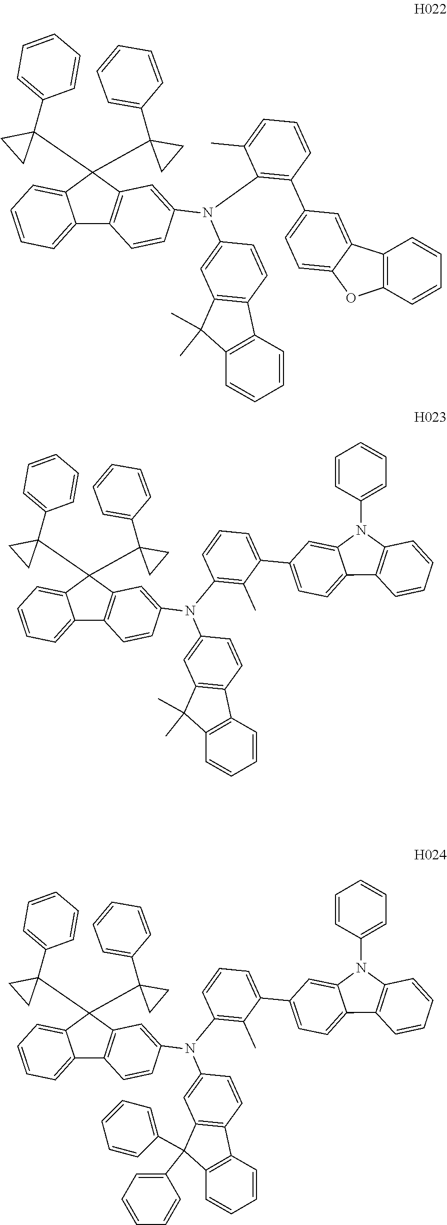 [SMILES: Cc1cccc(-c2ccc3oc4ccccc4c3c2)c1N(c1ccc2c(c1)C(C)(C)c1ccccc1-2)c1ccc2c(c1)C(C1(c3ccccc3)CC1)(C1(c3ccccc3)CC1)c1ccccc1-2 ; Cc1c(-c2ccc3c4ccccc4n(-c4ccccc4)c3c2)cccc1N(c1ccc2c(c1)C(C)(C)c1ccccc1-2)c1ccc2c(c1)C(C1(c3ccccc3)CC1)(C1(c3ccccc3)CC1)c1ccccc1-2 ; Cc1c(-c2ccc3c4ccccc4n(-c4ccccc4)c3c2)cccc1N(c1ccc2c(c1)C(c1ccccc1)(c1ccccc1)c1ccccc1-2)c1ccc2c(c1)C(C1(c3ccccc3)CC1)(C1(c3ccccc3)CC1)c1ccccc1-2]

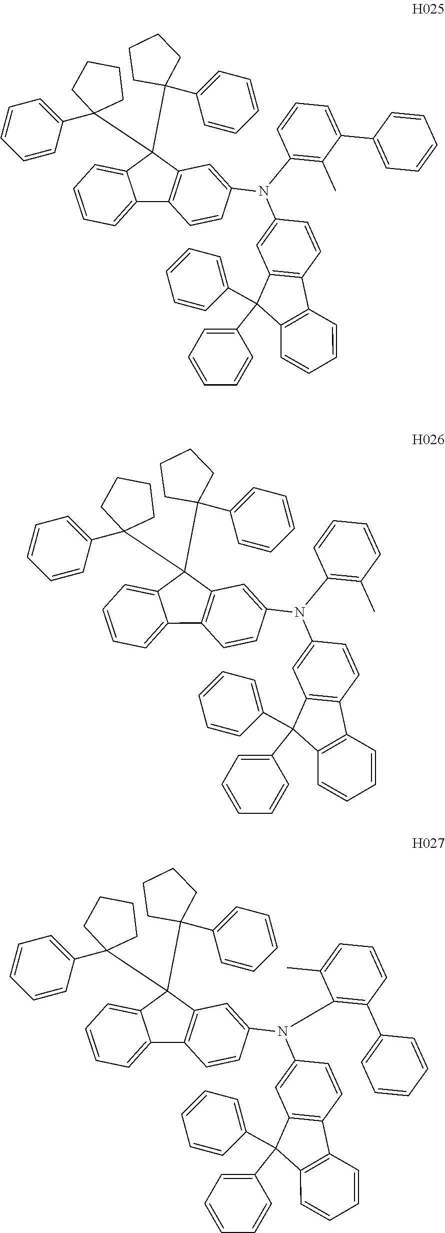 [SMILES: Cc1c(-c2ccccc2)cccc1N(c1ccc2c(c1)C(c1ccccc1)(c1ccccc1)c1ccccc1-2)c1ccc2c(c1)C(C1(c3ccccc3)CCCC1)(C1(c3ccccc3)CCCC1)c1ccccc1-2 ; Cc1ccccc1N(c1ccc2c(c1)C(c1ccccc1)(c1ccccc1)c1ccccc1-2)c1ccc2c(c1)C(C1(c3ccccc3)CCCC1)(C1(c3ccccc3)CCCC1)c1ccccc1-2 ; Cc1cccc(-c2ccccc2)c1N(c1ccc2c(c1)C(c1ccccc1)(c1ccccc1)c1ccccc1-2)c1ccc2c(c1)C(C1(c3ccccc3)CCCC1)(C1(c3ccccc3)CCCC1)c1ccccc1-2]

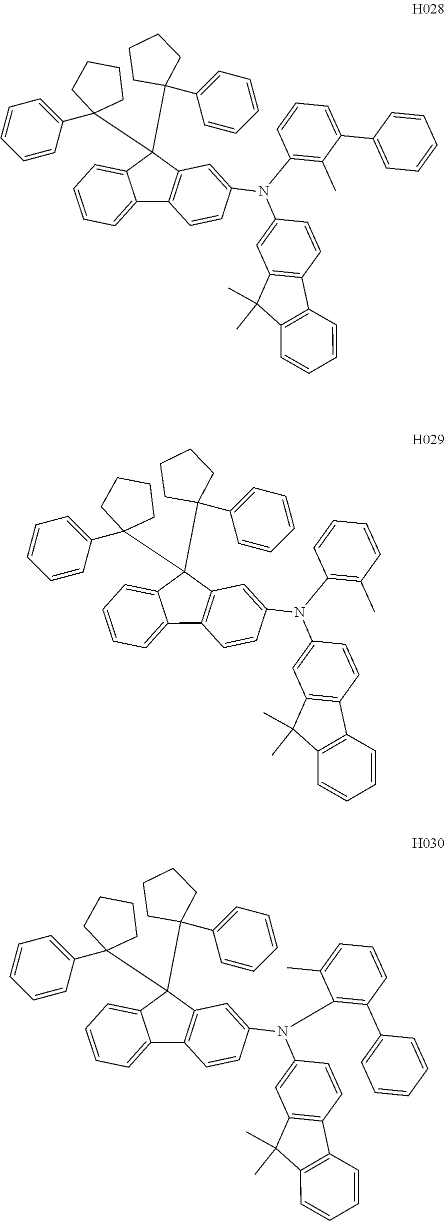 [SMILES: Cc1c(-c2ccccc2)cccc1N(c1ccc2c(c1)C(C)(C)c1ccccc1-2)c1ccc2c(c1)C(C1(c3ccccc3)CCCC1)(C1(c3ccccc3)CCCC1)c1ccccc1-2 ; Cc1ccccc1N(c1ccc2c(c1)C(C)(C)c1ccccc1-2)c1ccc2c(c1)C(C1(c3ccccc3)CCCC1)(C1(c3ccccc3)CCCC1)c1ccccc1-2 ; Cc1cccc(-c2ccccc2)c1N(c1ccc2c(c1)C(C)(C)c1ccccc1-2)c1ccc2c(c1)C(C1(c3ccccc3)CCCC1)(C1(c3ccccc3)CCCC1)c1ccccc1-2]

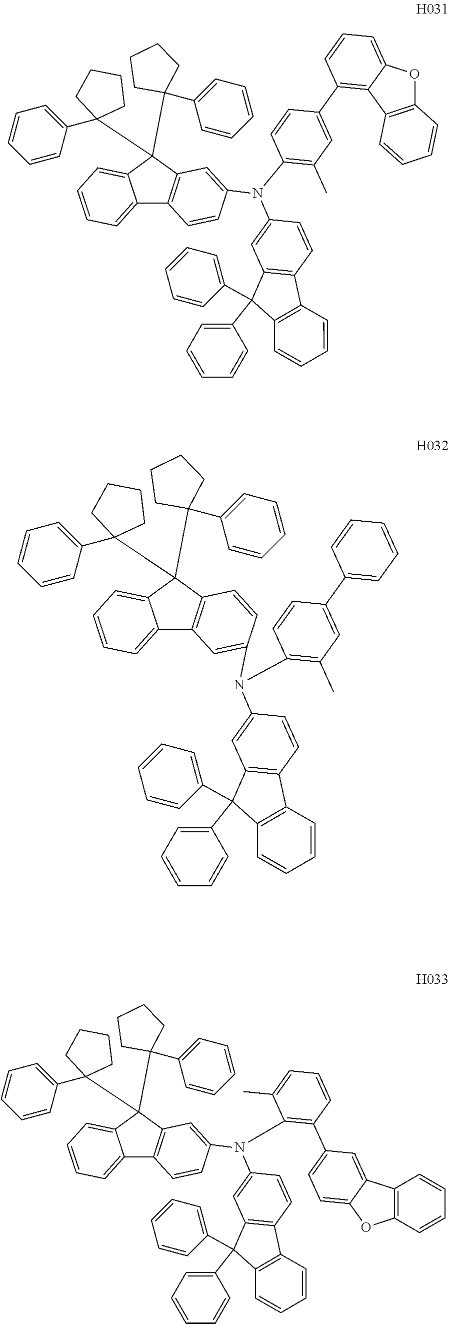 [SMILES: Cc1cc(-c2cccc3oc4ccccc4c23)ccc1N(c1ccc2c(c1)C(c1ccccc1)(c1ccccc1)c1ccccc1-2)c1ccc2c(c1)C(C1(c3ccccc3)CCCC1)(C1(c3ccccc3)CCCC1)c1ccccc1-2 ; Cc1cc(-c2ccccc2)ccc1N(c1ccc2c(c1)-c1ccccc1C2(C1(c2ccccc2)CCCC1)C1(c2ccccc2)CCCC1)c1ccc2c(c1)C(c1ccccc1)(c1ccccc1)c1ccccc1-2 ; Cc1cccc(-c2ccc3oc4ccccc4c3c2)c1N(c1ccc2c(c1)C(c1ccccc1)(c1ccccc1)c1ccccc1-2)c1ccc2c(c1)C(C1(c3ccccc3)CCCC1)(C1(c3ccccc3)CCCC1)c1ccccc1-2]

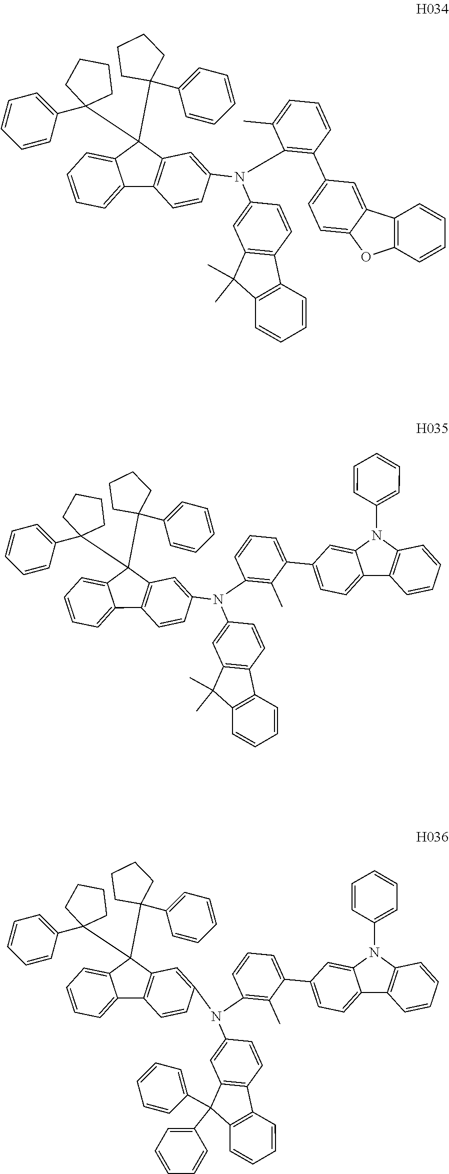 [SMILES: Cc1cccc(-c2ccc3oc4ccccc4c3c2)c1N(c1ccc2c(c1)C(C)(C)c1ccccc1-2)c1ccc2c(c1)C(C1(c3ccccc3)CCCC1)(C1(c3ccccc3)CCCC1)c1ccccc1-2 ; Cc1c(-c2ccc3c4ccccc4n(-c4ccccc4)c3c2)cccc1N(c1ccc2c(c1)C(C)(C)c1ccccc1-2)c1ccc2c(c1)C(C1(c3ccccc3)CCCC1)(C1(c3ccccc3)CCCC1)c1ccccc1-2 ; Cc1c(-c2ccc3c4ccccc4n(-c4ccccc4)c3c2)cccc1N(c1ccc2c(c1)C(c1ccccc1)(c1ccccc1)c1ccccc1-2)c1ccc2c(c1)C(C1(c3ccccc3)CCCC1)(C1(c3ccccc3)CCCC1)c1ccccc1-2]

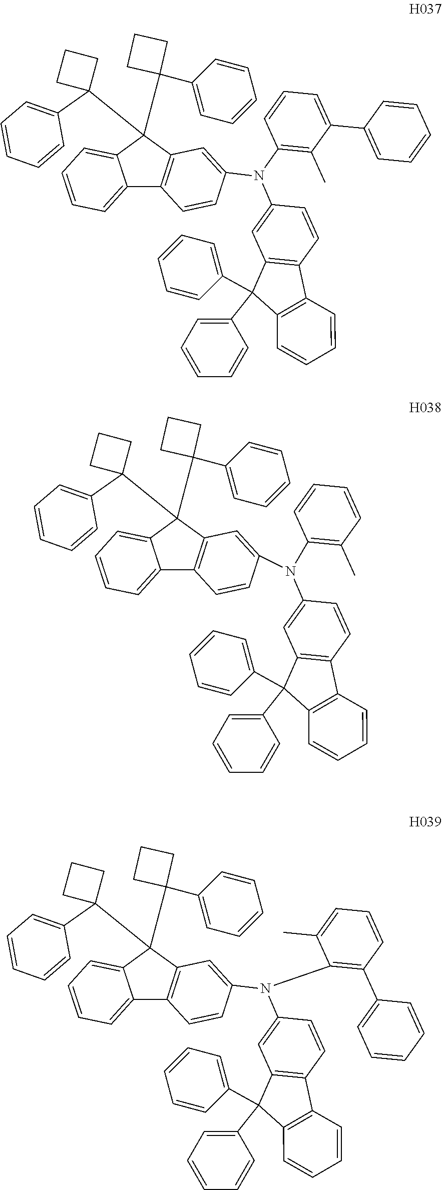 [SMILES: Cc1c(-c2ccccc2)cccc1N(c1ccc2c(c1)C(c1ccccc1)(c1ccccc1)c1ccccc1-2)c1ccc2c(c1)C(C1(c3ccccc3)CCC1)(C1(c3ccccc3)CCC1)c1ccccc1-2 ; Cc1ccccc1N(c1ccc2c(c1)C(c1ccccc1)(c1ccccc1)c1ccccc1-2)c1ccc2c(c1)C(C1(c3ccccc3)CCC1)(C1(c3ccccc3)CCC1)c1ccccc1-2 ; Cc1cccc(-c2ccccc2)c1N(c1ccc2c(c1)C(c1ccccc1)(c1ccccc1)c1ccccc1-2)c1ccc2c(c1)C(C1(c3ccccc3)CCC1)(C1(c3ccccc3)CCC1)c1ccccc1-2]

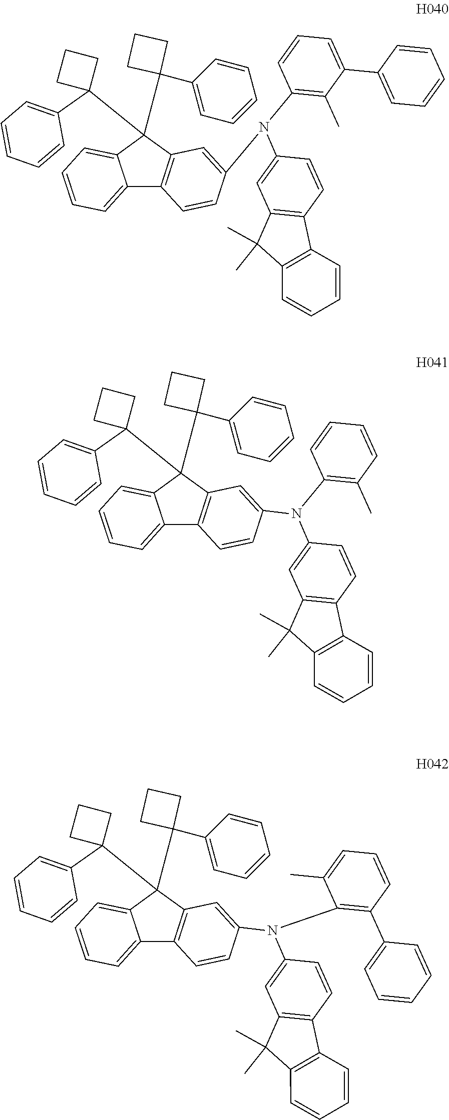 [SMILES: Cc1c(-c2ccccc2)cccc1N(c1ccc2c(c1)C(C)(C)c1ccccc1-2)c1ccc2c(c1)C(C1(c3ccccc3)CCC1)(C1(c3ccccc3)CCC1)c1ccccc1-2 ; Cc1ccccc1N(c1ccc2c(c1)C(C)(C)c1ccccc1-2)c1ccc2c(c1)C(C1(c3ccccc3)CCC1)(C1(c3ccccc3)CCC1)c1ccccc1-2 ; Cc1cccc(-c2ccccc2)c1N(c1ccc2c(c1)C(C)(C)c1ccccc1-2)c1ccc2c(c1)C(C1(c3ccccc3)CCC1)(C1(c3ccccc3)CCC1)c1ccccc1-2]

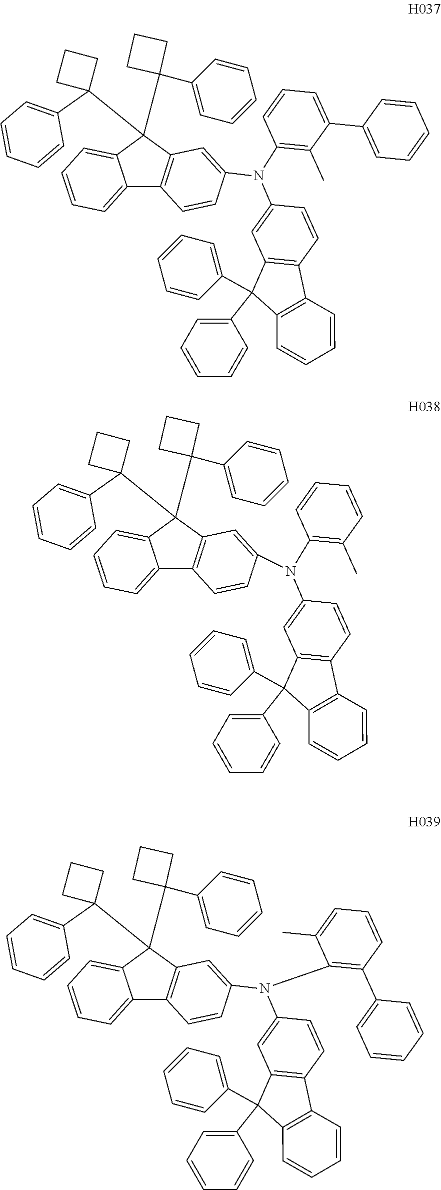 [SMILES: Cc1c(-c2ccccc2)cccc1N(c1ccc2c(c1)C(c1ccccc1)(c1ccccc1)c1ccccc1-2)c1ccc2c(c1)C(C1(c3ccccc3)CCC1)(C1(c3ccccc3)CCC1)c1ccccc1-2 ; Cc1ccccc1N(c1ccc2c(c1)C(c1ccccc1)(c1ccccc1)c1ccccc1-2)c1ccc2c(c1)C(C1(c3ccccc3)CCC1)(C1(c3ccccc3)CCC1)c1ccccc1-2 ; Cc1cccc(-c2ccccc2)c1N(c1ccc2c(c1)C(c1ccccc1)(c1ccccc1)c1ccccc1-2)c1ccc2c(c1)C(C1(c3ccccc3)CCC1)(C1(c3ccccc3)CCC1)c1ccccc1-2]

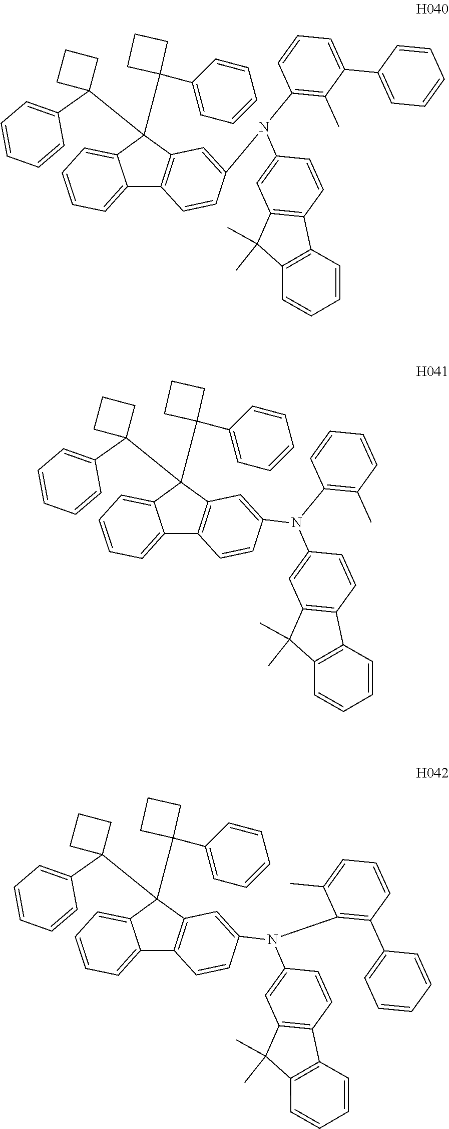 [SMILES: Cc1c(-c2ccccc2)cccc1N(c1ccc2c(c1)C(C)(C)c1ccccc1-2)c1ccc2c(c1)C(C1(c3ccccc3)CCC1)(C1(c3ccccc3)CCC1)c1ccccc1-2 ; Cc1ccccc1N(c1ccc2c(c1)C(C)(C)c1ccccc1-2)c1ccc2c(c1)C(C1(c3ccccc3)CCC1)(C1(c3ccccc3)CCC1)c1ccccc1-2 ; Cc1cccc(-c2ccccc2)c1N(c1ccc2c(c1)C(C)(C)c1ccccc1-2)c1ccc2c(c1)C(C1(c3ccccc3)CCC1)(C1(c3ccccc3)CCC1)c1ccccc1-2]

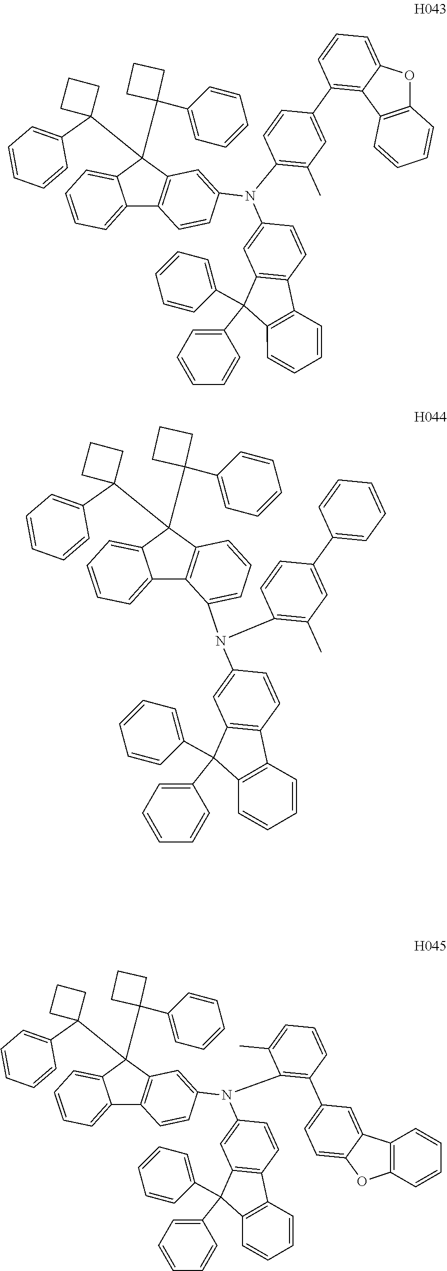 [SMILES: Cc1cc(-c2cccc3oc4ccccc4c23)ccc1N(c1ccc2c(c1)C(c1ccccc1)(c1ccccc1)c1ccccc1-2)c1ccc2c(c1)C(C1(c3ccccc3)CCC1)(C1(c3ccccc3)CCC1)c1ccccc1-2 ; Cc1cc(-c2ccccc2)ccc1N(c1ccc2c(c1)C(c1ccccc1)(c1ccccc1)c1ccccc1-2)c1cccc2c1-c1ccccc1C2(C1(c2ccccc2)CCC1)C1(c2ccccc2)CCC1 ; Cc1cccc(-c2ccc3oc4ccccc4c3c2)c1N(c1ccc2c(c1)C(c1ccccc1)(c1ccccc1)c1ccccc1-2)c1ccc2c(c1)C(C1(c3ccccc3)CCC1)(C1(c3ccccc3)CCC1)c1ccccc1-2]

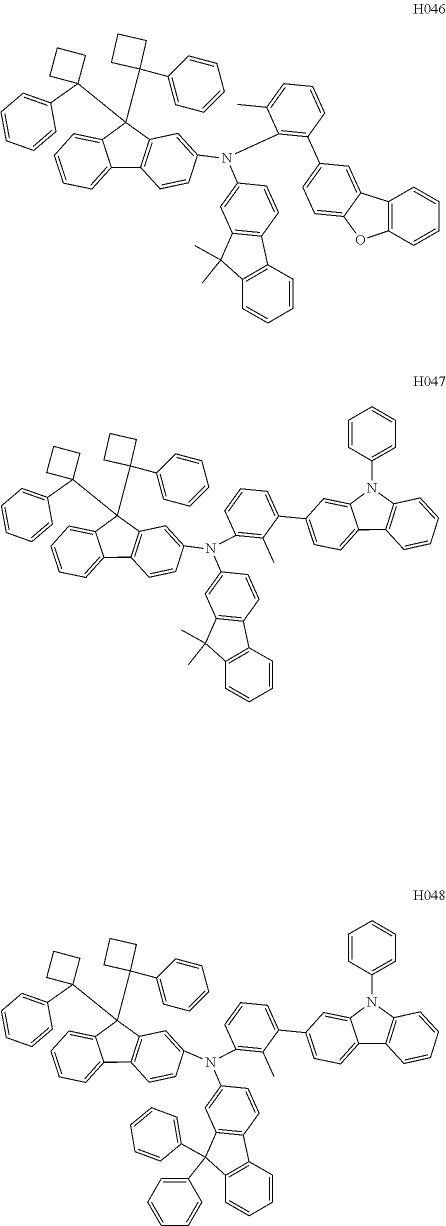 [SMILES: Cc1cccc(-c2ccc3oc4ccccc4c3c2)c1N(c1ccc2c(c1)C(C)(C)c1ccccc1-2)c1ccc2c(c1)C(C1(c3ccccc3)CCC1)(C1(c3ccccc3)CCC1)c1ccccc1-2 ; Cc1c(-c2ccc3c4ccccc4n(-c4ccccc4)c3c2)cccc1N(c1ccc2c(c1)C(C)(C)c1ccccc1-2)c1ccc2c(c1)C(C1(c3ccccc3)CCC1)(C1(c3ccccc3)CCC1)c1ccccc1-2 ; Cc1c(-c2ccc3c4ccccc4n(-c4ccccc4)c3c2)cccc1N(c1ccc2c(c1)C(c1ccccc1)(c1ccccc1)c1ccccc1-2)c1ccc2c(c1)C(C1(c3ccccc3)CCC1)(C1(c3ccccc3)CCC1)c1ccccc1-2]

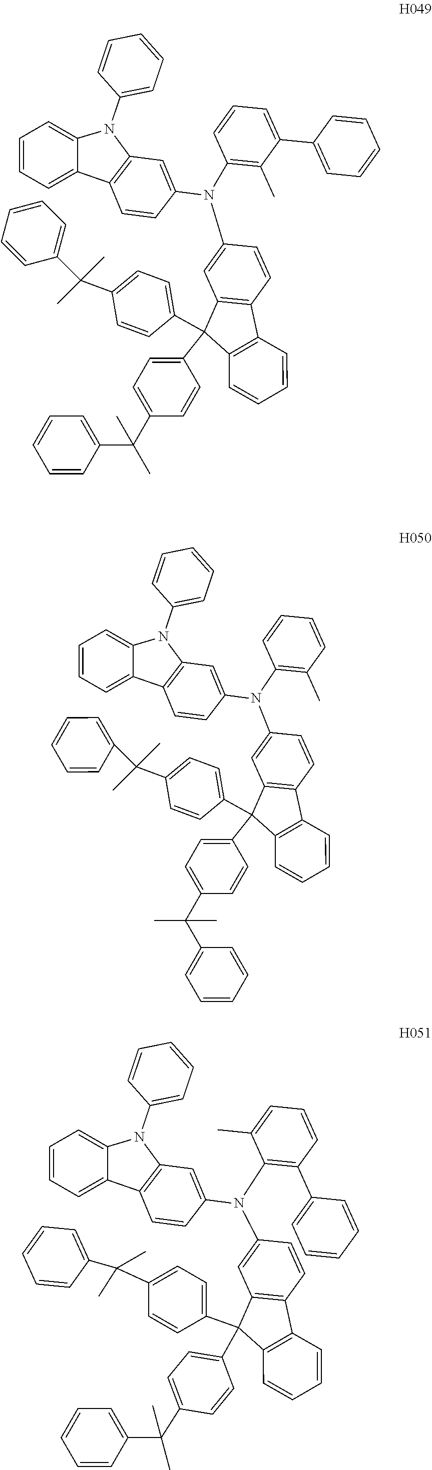 [SMILES: Cc1c(-c2ccccc2)cccc1N(c1ccc2c(c1)C(c1ccc(C(C)(C)c3ccccc3)cc1)(c1ccc(C(C)(C)c3ccccc3)cc1)c1ccccc1-2)c1ccc2c3ccccc3n(-c3ccccc3)c2c1 ; Cc1ccccc1N(c1ccc2c(c1)C(c1ccc(C(C)(C)c3ccccc3)cc1)(c1ccc(C(C)(C)c3ccccc3)cc1)c1ccccc1-2)c1ccc2c3ccccc3n(-c3ccccc3)c2c1 ; Cc1cccc(-c2ccccc2)c1N(c1ccc2c(c1)C(c1ccc(C(C)(C)c3ccccc3)cc1)(c1ccc(C(C)(C)c3ccccc3)cc1)c1ccccc1-2)c1ccc2c3ccccc3n(-c3ccccc3)c2c1]

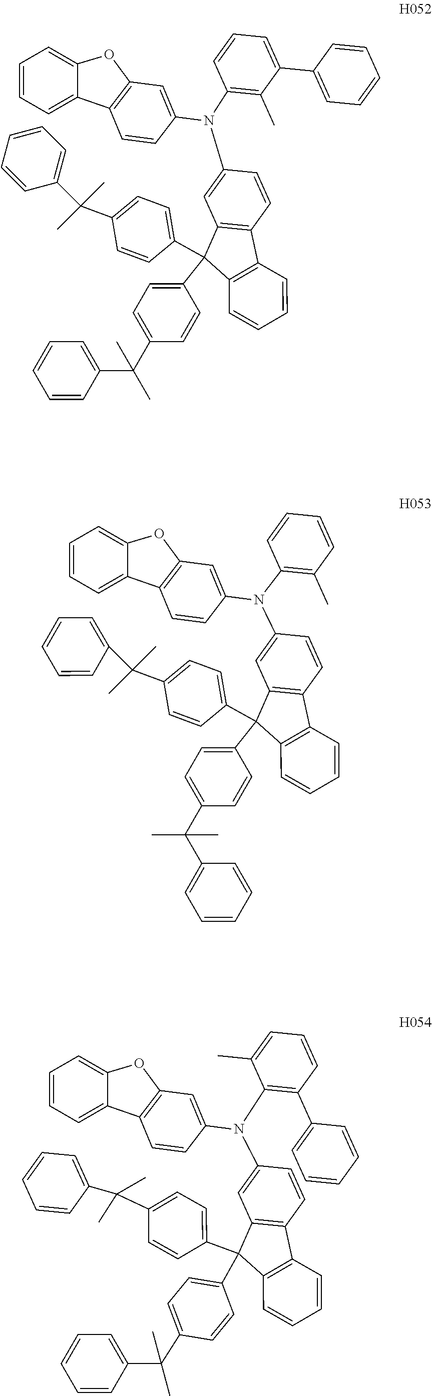 [SMILES: Cc1c(-c2ccccc2)cccc1N(c1ccc2c(c1)C(c1ccc(C(C)(C)c3ccccc3)cc1)(c1ccc(C(C)(C)c3ccccc3)cc1)c1ccccc1-2)c1ccc2c(c1)oc1ccccc12 ; Cc1ccccc1N(c1ccc2c(c1)C(c1ccc(C(C)(C)c3ccccc3)cc1)(c1ccc(C(C)(C)c3ccccc3)cc1)c1ccccc1-2)c1ccc2c(c1)oc1ccccc12 ; Cc1cccc(-c2ccccc2)c1N(c1ccc2c(c1)C(c1ccc(C(C)(C)c3ccccc3)cc1)(c1ccc(C(C)(C)c3ccccc3)cc1)c1ccccc1-2)c1ccc2c(c1)oc1ccccc12]

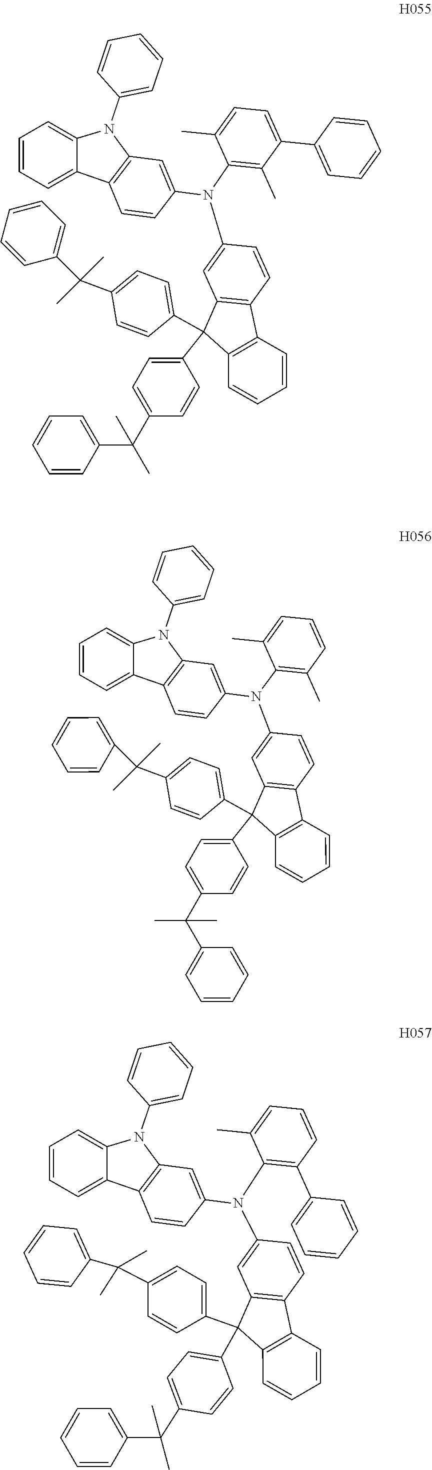 [SMILES: Cc1ccc(-c2ccccc2)c(C)c1N(c1ccc2c(c1)C(c1ccc(C(C)(C)c3ccccc3)cc1)(c1ccc(C(C)(C)c3ccccc3)cc1)c1ccccc1-2)c1ccc2c3ccccc3n(-c3ccccc3)c2c1 ; Cc1cccc(C)c1N(c1ccc2c(c1)C(c1ccc(C(C)(C)c3ccccc3)cc1)(c1ccc(C(C)(C)c3ccccc3)cc1)c1ccccc1-2)c1ccc2c3ccccc3n(-c3ccccc3)c2c1 ; Cc1cccc(-c2ccccc2)c1N(c1ccc2c(c1)C(c1ccc(C(C)(C)c3ccccc3)cc1)(c1ccc(C(C)(C)c3ccccc3)cc1)c1ccccc1-2)c1ccc2c3ccccc3n(-c3ccccc3)c2c1]

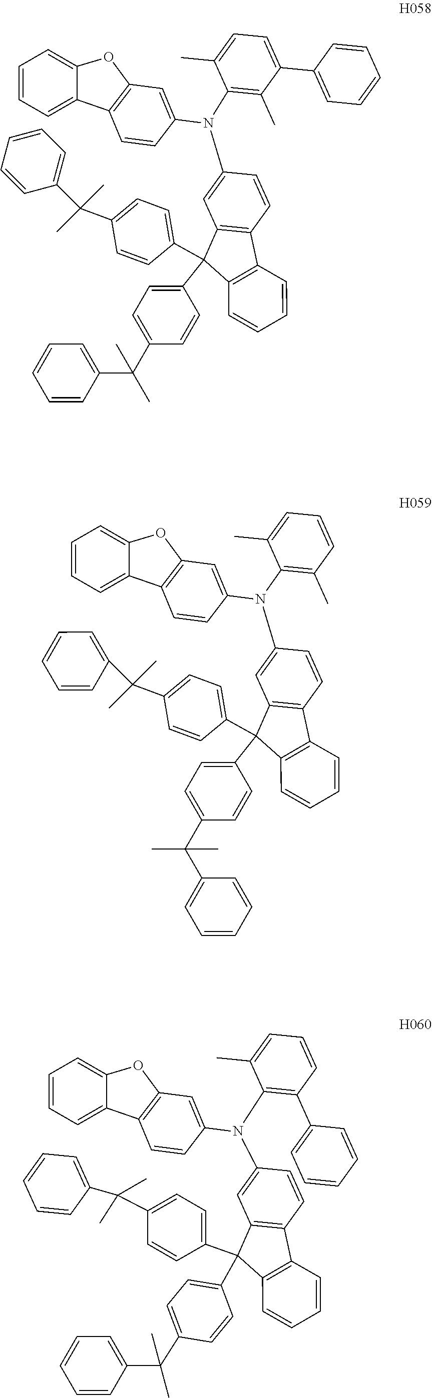 [SMILES: Cc1ccc(-c2ccccc2)c(C)c1N(c1ccc2c(c1)C(c1ccc(C(C)(C)c3ccccc3)cc1)(c1ccc(C(C)(C)c3ccccc3)cc1)c1ccccc1-2)c1ccc2c(c1)oc1ccccc12 ; Cc1cccc(C)c1N(c1ccc2c(c1)C(c1ccc(C(C)(C)c3ccccc3)cc1)(c1ccc(C(C)(C)c3ccccc3)cc1)c1ccccc1-2)c1ccc2c(c1)oc1ccccc12 ; Cc1cccc(-c2ccccc2)c1N(c1ccc2c(c1)C(c1ccc(C(C)(C)c3ccccc3)cc1)(c1ccc(C(C)(C)c3ccccc3)cc1)c1ccccc1-2)c1ccc2c(c1)oc1ccccc12]

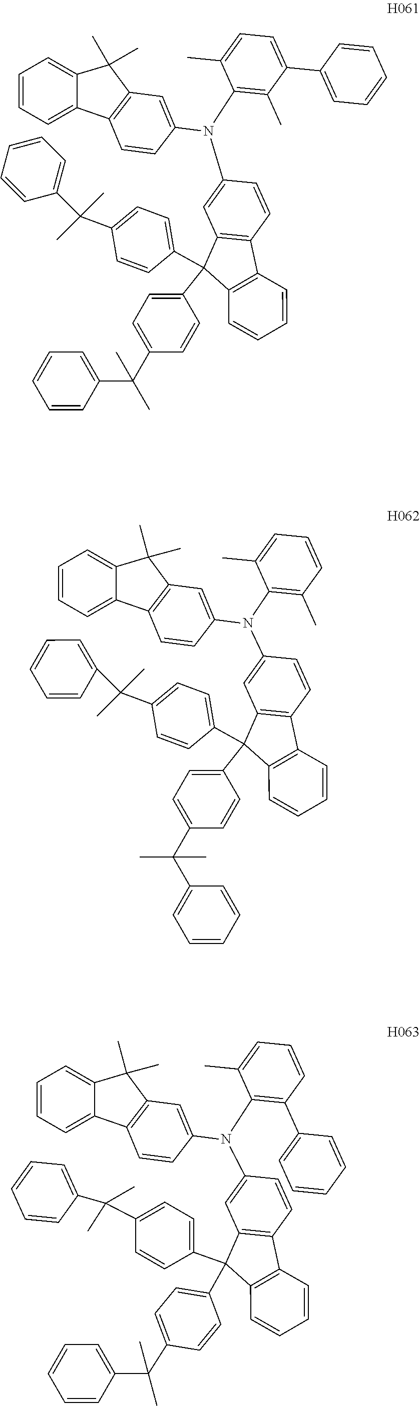 [SMILES: Cc1ccc(-c2ccccc2)c(C)c1N(c1ccc2c(c1)C(C)(C)c1ccccc1-2)c1ccc2c(c1)C(c1ccc(C(C)(C)c3ccccc3)cc1)(c1ccc(C(C)(C)c3ccccc3)cc1)c1ccccc1-2 ; Cc1cccc(C)c1N(c1ccc2c(c1)C(C)(C)c1ccccc1-2)c1ccc2c(c1)C(c1ccc(C(C)(C)c3ccccc3)cc1)(c1ccc(C(C)(C)c3ccccc3)cc1)c1ccccc1-2 ; Cc1cccc(-c2ccccc2)c1N(c1ccc2c(c1)C(C)(C)c1ccccc1-2)c1ccc2c(c1)C(c1ccc(C(C)(C)c3ccccc3)cc1)(c1ccc(C(C)(C)c3ccccc3)cc1)c1ccccc1-2]

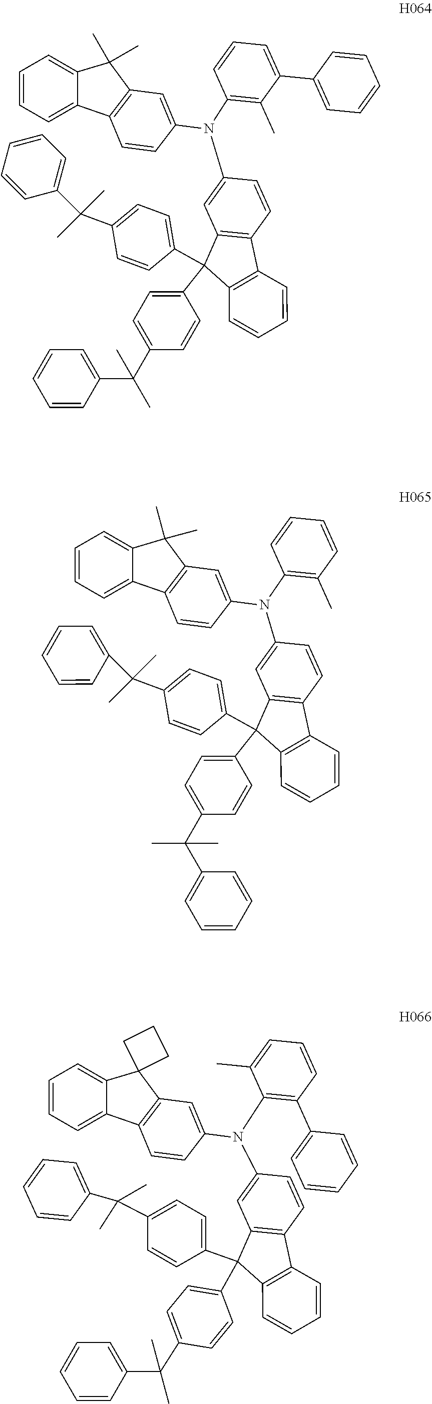 [SMILES: Cc1c(-c2ccccc2)cccc1N(c1ccc2c(c1)C(C)(C)c1ccccc1-2)c1ccc2c(c1)C(c1ccc(C(C)(C)c3ccccc3)cc1)(c1ccc(C(C)(C)c3ccccc3)cc1)c1ccccc1-2 ; Cc1ccccc1N(c1ccc2c(c1)C(C)(C)c1ccccc1-2)c1ccc2c(c1)C(c1ccc(C(C)(C)c3ccccc3)cc1)(c1ccc(C(C)(C)c3ccccc3)cc1)c1ccccc1-2 ; Cc1cccc(-c2ccccc2)c1N(c1ccc2c(c1)C1(CCC1)c1ccccc1-2)c1ccc2c(c1)C(c1ccc(C(C)(C)c3ccccc3)cc1)(c1ccc(C(C)(C)c3ccccc3)cc1)c1ccccc1-2]

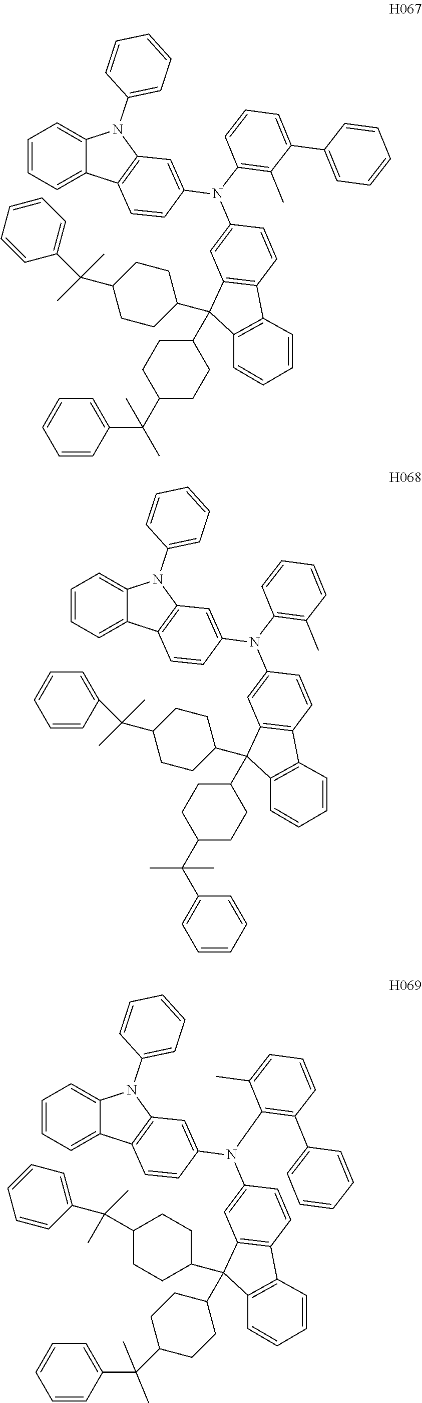

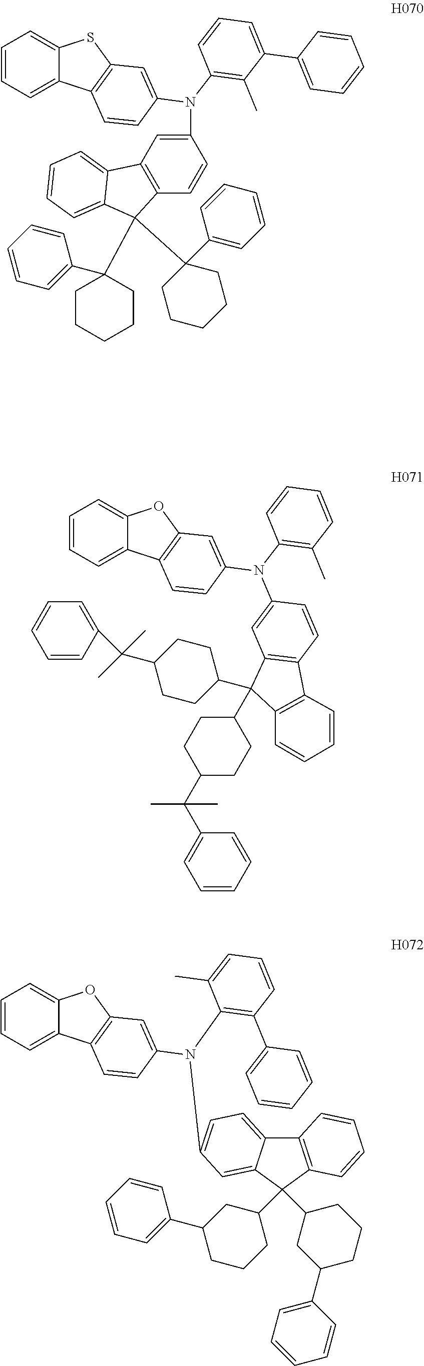 [SMILES: Cc1c(-c2ccccc2)cccc1N(c1ccc2c(c1)-c1ccccc1C2(C1(c2ccccc2)CCCCC1)C1(c2ccccc2)CCCCC1)c1ccc2c(c1)sc1ccccc12 ; Cc1ccccc1N(c1ccc2c(c1)C(C1CCC(C(C)(C)c3ccccc3)CC1)(C1CCC(C(C)(C)c3ccccc3)CC1)c1ccccc1-2)c1ccc2c(c1)oc1ccccc12 ; Cc1cccc(-c2ccccc2)c1N(c1ccc2c(c1)C(C1CCCC(c3ccccc3)C1)(C1CCCC(c3ccccc3)C1)c1ccccc1-2)c1ccc2c(c1)oc1ccccc12]

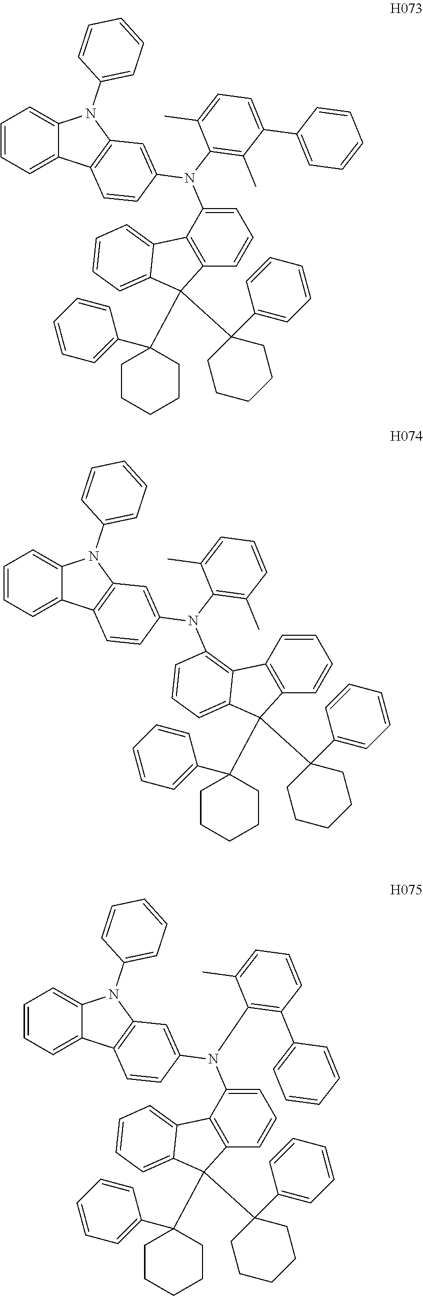 [SMILES: Cc1ccc(-c2ccccc2)c(C)c1N(c1ccc2c3ccccc3n(-c3ccccc3)c2c1)c1cccc2c1-c1ccccc1C2(C1(c2ccccc2)CCCCC1)C1(c2ccccc2)CCCCC1 ; Cc1cccc(C)c1N(c1ccc2c3ccccc3n(-c3ccccc3)c2c1)c1cccc2c1-c1ccccc1C2(C1(c2ccccc2)CCCCC1)C1(c2ccccc2)CCCCC1 ; Cc1cccc(-c2ccccc2)c1N(c1ccc2c3ccccc3n(-c3ccccc3)c2c1)c1cccc2c1-c1ccccc1C2(C1(c2ccccc2)CCCCC1)C1(c2ccccc2)CCCCC1]

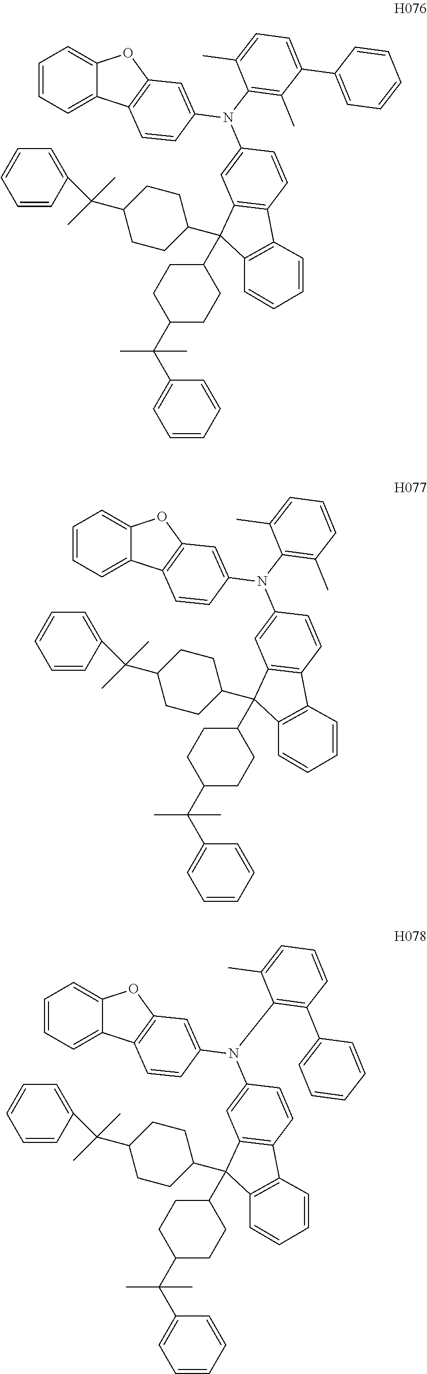 [SMILES: Cc1ccc(-c2ccccc2)c(C)c1N(c1ccc2c(c1)C(C1CCC(C(C)(C)c3ccccc3)CC1)(C1CCC(C(C)(C)c3ccccc3)CC1)c1ccccc1-2)c1ccc2c(c1)oc1ccccc12 ; Cc1cccc(C)c1N(c1ccc2c(c1)C(C1CCC(C(C)(C)c3ccccc3)CC1)(C1CCC(C(C)(C)c3ccccc3)CC1)c1ccccc1-2)c1ccc2c(c1)oc1ccccc12 ; Cc1cccc(-c2ccccc2)c1N(c1ccc2c(c1)C(C1CCC(C(C)(C)c3ccccc3)CC1)(C1CCC(C(C)(C)c3ccccc3)CC1)c1ccccc1-2)c1ccc2c(c1)oc1ccccc12]

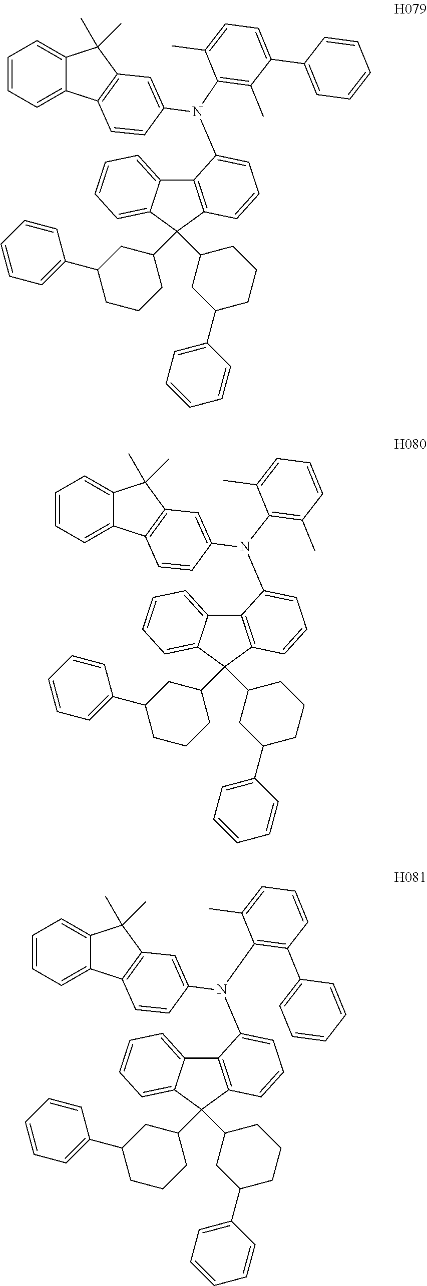 [SMILES: Cc1ccc(-c2ccccc2)c(C)c1N(c1ccc2c(c1)C(C)(C)c1ccccc1-2)c1cccc2c1-c1ccccc1C2(C1CCCC(c2ccccc2)C1)C1CCCC(c2ccccc2)C1 ; Cc1cccc(C)c1N(c1ccc2c(c1)C(C)(C)c1ccccc1-2)c1cccc2c1-c1ccccc1C2(C1CCCC(c2ccccc2)C1)C1CCCC(c2ccccc2)C1 ; Cc1cccc(-c2ccccc2)c1N(c1ccc2c(c1)C(C)(C)c1ccccc1-2)c1cccc2c1-c1ccccc1C2(C1CCCC(c2ccccc2)C1)C1CCCC(c2ccccc2)C1]

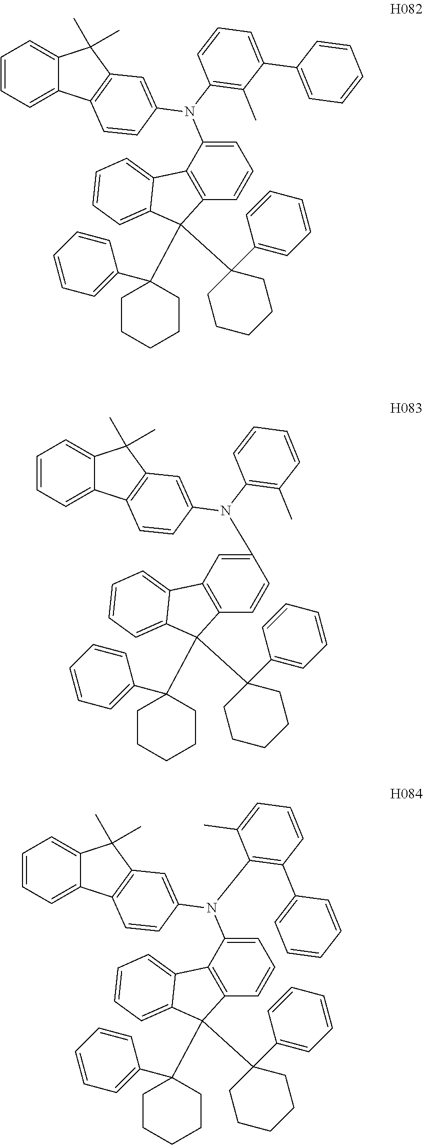 [SMILES: Cc1c(-c2ccccc2)cccc1N(c1ccc2c(c1)C(C)(C)c1ccccc1-2)c1cccc2c1-c1ccccc1C2(C1(c2ccccc2)CCCCC1)C1(c2ccccc2)CCCCC1 ; Cc1ccccc1N(c1ccc2c(c1)-c1ccccc1C2(C1(c2ccccc2)CCCCC1)C1(c2ccccc2)CCCCC1)c1ccc2c(c1)C(C)(C)c1ccccc1-2 ; Cc1cccc(-c2ccccc2)c1N(c1ccc2c(c1)C(C)(C)c1ccccc1-2)c1cccc2c1-c1ccccc1C2(C1(c2ccccc2)CCCCC1)C1(c2ccccc2)CCCCC1]

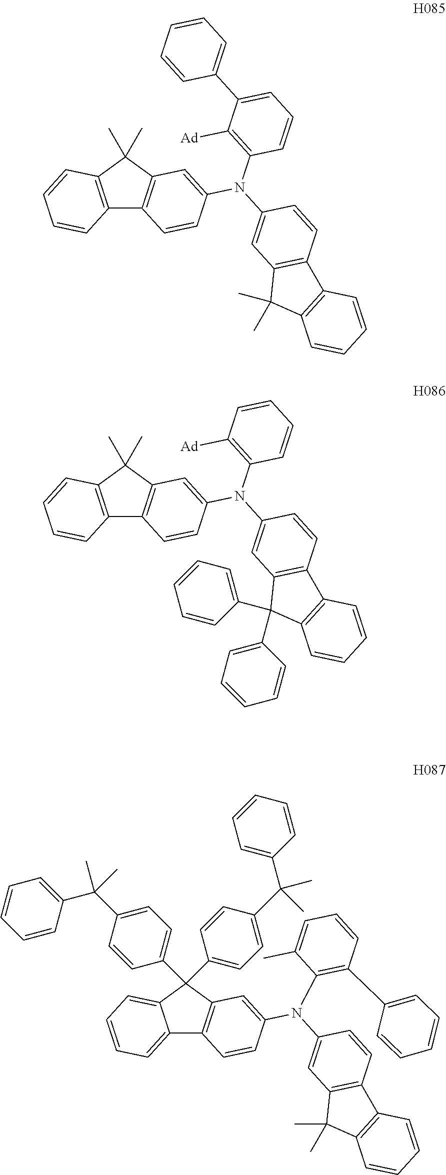

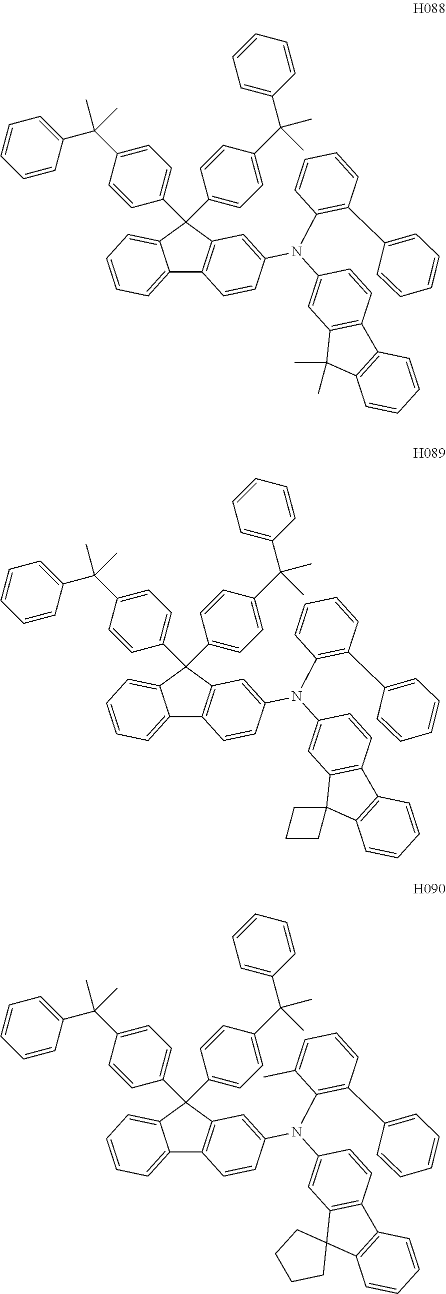 [SMILES: CC(C)(c1ccccc1)c1ccc(C2(c3ccc(C(C)(C)c4ccccc4)cc3)c3ccccc3-c3ccc(N(c4ccc5c(c4)C(C)(C)c4ccccc4-5)c4ccccc4-c4ccccc4)cc32)cc1 ; CC(C)(c1ccccc1)c1ccc(C2(c3ccc(C(C)(C)c4ccccc4)cc3)c3ccccc3-c3ccc(N(c4ccc5c(c4)C4(CCC4)c4ccccc4-5)c4ccccc4-c4ccccc4)cc32)cc1 ; Cc1cccc(-c2ccccc2)c1N(c1ccc2c(c1)C1(CCCC1)c1ccccc1-2)c1ccc2c(c1)C(c1ccc(C(C)(C)c3ccccc3)cc1)(c1ccc(C(C)(C)c3ccccc3)cc1)c1ccccc1-2]

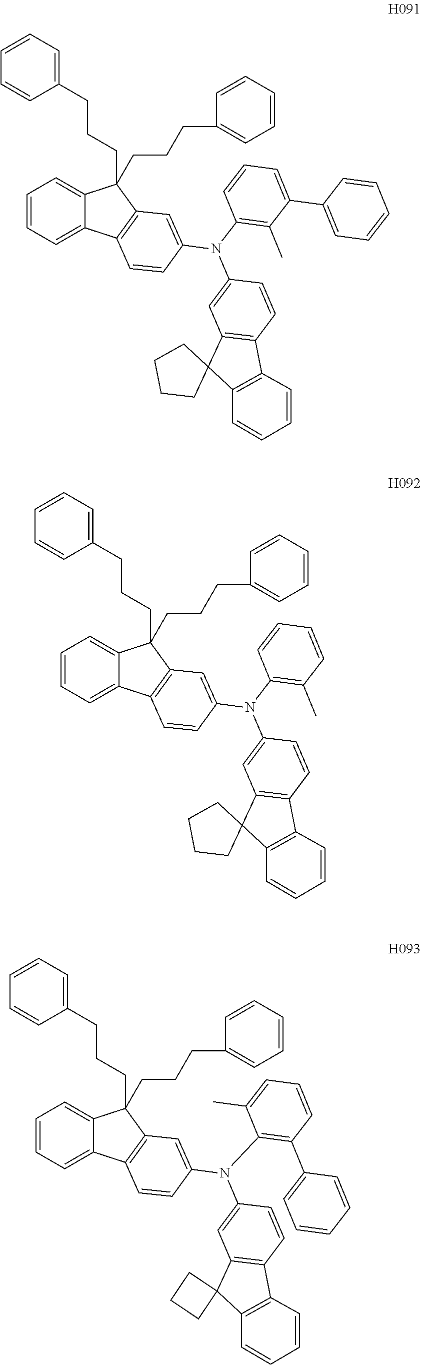 [SMILES: Cc1c(-c2ccccc2)cccc1N(c1ccc2c(c1)C1(CCCC1)c1ccccc1-2)c1ccc2c(c1)C(CCCc1ccccc1)(CCCc1ccccc1)c1ccccc1-2 ; Cc1ccccc1N(c1ccc2c(c1)C1(CCCC1)c1ccccc1-2)c1ccc2c(c1)C(CCCc1ccccc1)(CCCc1ccccc1)c1ccccc1-2 ; Cc1cccc(-c2ccccc2)c1N(c1ccc2c(c1)C(CCCc1ccccc1)(CCCc1ccccc1)c1ccccc1-2)c1ccc2c(c1)C1(CCC1)c1ccccc1-2]

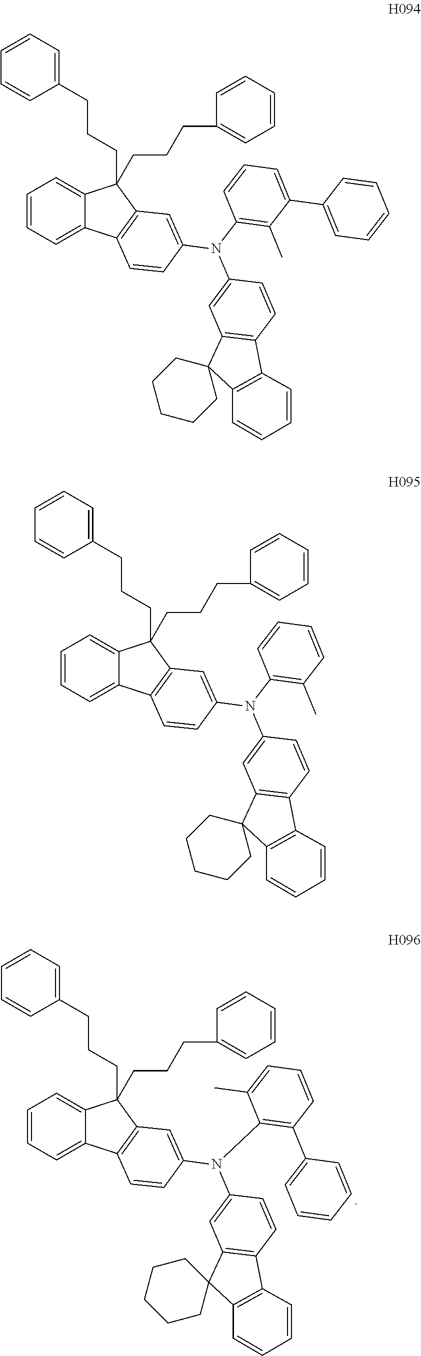 [SMILES: Cc1c(-c2ccccc2)cccc1N(c1ccc2c(c1)C1(CCCCC1)c1ccccc1-2)c1ccc2c(c1)C(CCCc1ccccc1)(CCCc1ccccc1)c1ccccc1-2 ; Cc1ccccc1N(c1ccc2c(c1)C1(CCCCC1)c1ccccc1-2)c1ccc2c(c1)C(CCCc1ccccc1)(CCCc1ccccc1)c1ccccc1-2 ; Cc1cccc(-c2ccccc2)c1N(c1ccc2c(c1)C1(CCCCC1)c1ccccc1-2)c1ccc2c(c1)C(CCCc1ccccc1)(CCCc1ccccc1)c1ccccc1-2]

Here, Ad represents an adamantyl group.

Embodiments of the present disclosure further provide an application of the hole transport material as described above in an organic electroluminescent device.

Embodiments of the present er provide an organic electroluminescent device. The organic electroluminescent device includes:
a substrate;
a first electrode, which is disposed on the substrate;
an organic light-emitting functional layer, which is disposed on the first electrode; and
a second electrode, which is disposed on the organic light-emitting functional layer.

The organic light-emitting functional layer includes a hole transport layer. The hole transport layer includes the hole transport material as described above.

Embodiments of the present disclosure further provide a composition. The composition contains the hole transport material as described above.

Embodiments of the present disclosure further provide a formulation. The formulation contains the hole transport material as described above and at least one solvent, or contains the composition as described above and at least one solvent. The solvent is not particularly limited, and may be used, for example, an unsaturated hydrocarbon solvent such as toluene, xylene, mesitylene, tetralin, decahydronaphthalene, dicyclohexane, n-butylbenzene, sec-butylbenzene or tert-butylbenzene, or a halogenated saturated hydrocarbon solvent such as carbon tetrachloride, chloroform, dichloromethane, dichloroethane, chlorobutane, bromobutane, chloropentane, bromopentane, chlorohexane, bromohexane, chlorocyclohexane or bromocyclohexane, or a halogenated unsaturated hydrocarbon solvent such as chlorobenzene, dichlorobenzene or trichlorobenzene, or an ether solvent such as tetrahydrofuran or tetrahydropyran, or an ester solvent such as alkyl benzoate, as well known to those skilled in the art.

The organic electroluminescent device in the embodiments of the present disclosure may be used in an OLED lighting or display apparatus.

Embodiments of the present disclosure further provide a display or lighting apparatus. The apparatus includes one or more of the organic electroluminescent devices as described above.

In summary, compared with the prior art, the embodiments of the present disclosure have the beneficial effects below:
the hole transport material provided in the embodiments of the present disclosure uses triarylamine as a parent nucleus, and uses an aryl and alkyl co-substituted fluorenyl group cooperated with a heteroaryl group and an aryl group on substitution sites of other side chains, so that the hole transport material has an excellent light-emitting efficiency and a good evaporation stability. In addition, when the hole transport material provided in the embodiments of the present disclosure is used in an organic electroluminescent device, it is conducive to improving a hole transport efficiency of the organic electroluminescent device, thereby effectively improving a light-emitting efficiency and a working life of the organic electroluminescent device.

### BRIEF DESCRIPTION OF THE DRAWINGS

In order to describe technical solutions in the present disclosure more clearly, accompanying drawings to be used in some embodiments of the present disclosure will be introduced briefly below. However, the accompanying drawings to be described below are merely accompanying drawings of some embodiments of the present disclosure, and a person having ordinary skill in the art can obtain other drawings according to these accompanying drawings. In addition, the accompanying drawings in the following description may be regarded as schematic diagrams, and are not limitations to actual sizes of products, actual processes of methods or actual timings of signals to which the embodiments of the present disclosure relate.

FIG. 1 is a structural diagram of an organic electroluminescent device in accordance with some embodiments of the present disclosure;

FIG. 2 is a structural diagram of an organic light-emitting diode (OLED) in accordance with some embodiments of the present disclosure; and

FIG. 3 is a structural diagram of another OLED in accordance with some embodiments of the present disclosure.

### DETAILED DESCRIPTION

Technical solutions of the present disclosure will be described clearly and completely by using embodiments below. However, the described embodiments are merely some but not all embodiments of the present disclosure. On the basis of the embodiments of the present disclosure, all other embodiments obtained by a person of skilled in the art without creative labour shall be included in the protection scope of the present disclosure.

An aryl group as used in the embodiments of the present disclosure is a generic term for monovalent groups remaining after removal of one hydrogen atom from an aromatic nucleus carbon of an aromatic hydrocarbon molecule. The aryl group may be a single ring aryl group or a fused ring aryl group. For the aryl group or an aromatic group, as used herein, a non-fused system and a fused system are considered. The aryl group may be, but is not limited to, an aryl group having 6 to 30 carbon atoms. For example, the aryl group is an aryl group having 6 to 20 carbon atoms. For example, the aryl group is an aryl group having 6 to 12 carbon atoms. Examples of the aryl group include a phenyl group, a biphenyl group, a terphenyl group, a triphenylenyl group, a tetraphenylene group, a naphthyl group, an anthryl group, a phenalene group, a phenanthryl group, a fluorenyl group, a pyrenyl group, a perylenyl group and an azulenyl group. For example, the aryl group is a phenyl group, a biphenyl group, a terphenyl group, a triphenylenyl group, a fluorenyl group or a naphthyl group. Examples of a non-fused aryl group include a phenyl group, a biphenyl-2-yl group, a biphenyl-3-yl group, a biphenyl-4-yl group, a p-terphenyl-4-yl group, a p-terphenyl-3-yl group, a p-terphenyl-2-yl group, an m-terphenyl-4-yl group, an m-terphenyl-3-yl group, an m-terphenyl-2-yl group, an o-tolyl group, an m-tolyl group, a p-tolyl group, a p-(2-phenylpropyl)phenyl group, a 4′-methylbiphenyl group, a 4″-tert-butyl-p-terphenyl-4-yl group, an o-cumyl group, an m-cumyl group, a p-cumyl group, a 2,3-dimethylphenyl group, a 3,4-dimethylphenyl group, a 2,5-dimethylphenyl group, a mesityl group and a m-quaterphenyl group.

A heteroaryl group as used in the embodiments of the present disclosure is a generic term for groups obtained by substitution of one or more aromatic nucleus carbons in an aryl group by heteroatoms, a heteroatom therein including but not being limited to an oxygen atom, a sulfur atom, a silicon atom or a nitrogen atom. The heteroaryl group may be a single ring heteroaryl group or a fused ring heteroaryl group, and may be a heteroaryl group having 6 to 30 carbon atoms. For example, the heteroaryl group is a heteroaryl group having 6 to 20 carbon atoms. Examples of the heteroaryl group may include, but are not limited to, a pyridyl group, a pyrrolyl group, a thienyl group, a furanyl group, an indolyl group, a quinolyl group, an isoquinolyl group, a benzothienyl group, a benzofuranyl group, a dibenzofuranyl group, a dibenzothienyl group, a carbazolyl group, etc.

Alkyl groups as used in the embodiments of the present disclosure include a linear alkyl group and a branched alkyl group. An alkyl group may be an alkyl group having 1 to 24 carbon atoms. For example, the alkyl group is an alkyl group having 1 to 10 carbon atoms. Examples of the alkyl group includes a methyl group, an ethyl group, a propyl group, 1-methylethyl group, a butyl group, a 1-methylpropyl group, a 2-methylpropyl group, a pentyl group, a 1-methylbutyl group, a 2-methylbutyl group, a 3-methylbutyl group, a 1,1-dimethylpropyl group, a 1,2-dimethylpropyl group, a 2,2-dimethylpropyl group, etc. In addition, the alkyl group may optionally be substituted.

A cycloalkyl group as used in the embodiments of the present disclosure represents any functional group or substituent derived from an alicyclic ring. For example, the cycloalkyl group may be a saturated cycloalkyl group having more than or equal to 3 and less than or equal to 20 ring carbon atoms. The cycloalkyl group may be monocyclic, annelated, bridged cyclic, spirocyclic or fused cyclic. For example, the cycloalkyl group may be a cyclohexane group, a bicyclohexyl group, an adamantyl group, a bicyclo[2.2.2]octane group, a norbornanyl group, etc., which is not limited thereto.

Throughout the description, unless explicitly described to the contrary, the expression “including/comprising” any component shall be construed as implying the inclusion of other element(s), but not the exclusion of any other element. In addition, it will be understood that, throughout the description, when an element such as a layer, a film, a region or a substrate is described as being “on” or “above” another element, the element may be “directly on” the another element, or there may be intermediate element(s) between the element and the another element. Furthermore, the term “on” or “above” means being located on a side of a target portion, but does not necessarily mean being located above the target portion in a direction of gravity.

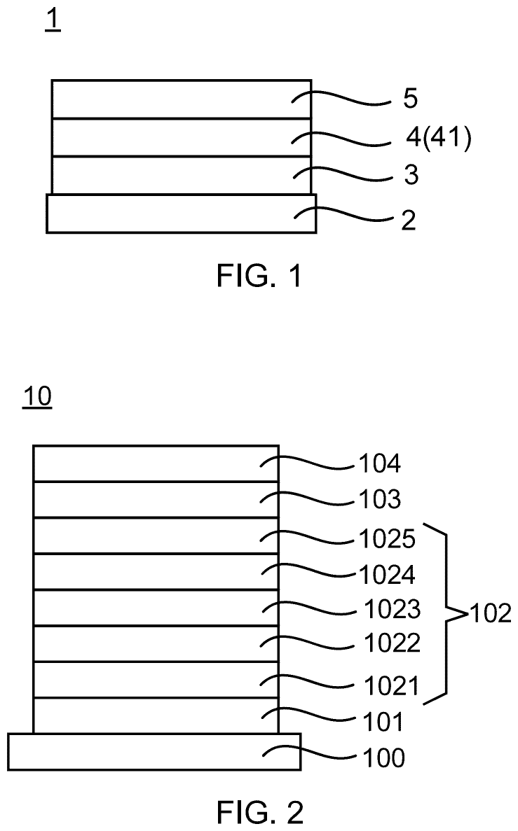

A purpose of embodiments of the present disclosure is to provide an organic electroluminescent device. With reference to FIG. 1, the organic electroluminescent device 1 includes: a substrate 2, a first electrode 3 disposed on the substrate 2, an organic light-emitting functional layer 4 disposed on the first electrode 3, and a second electrode 5 disposed on the organic light-emitting functional layer 4. The organic light-emitting functional layer 4 includes a hole transport layer 41. The hole transport layer 41 contains a hole transport material as shown in formula (I).

In one implementation of the present disclosure, the hole transport layer of the organic electroluminescent device (also referred to as an organic light-emitting diode, which is abbreviated as an OLED) includes one or more compounds as shown in general formula (I) above to serve as the hole transport material.

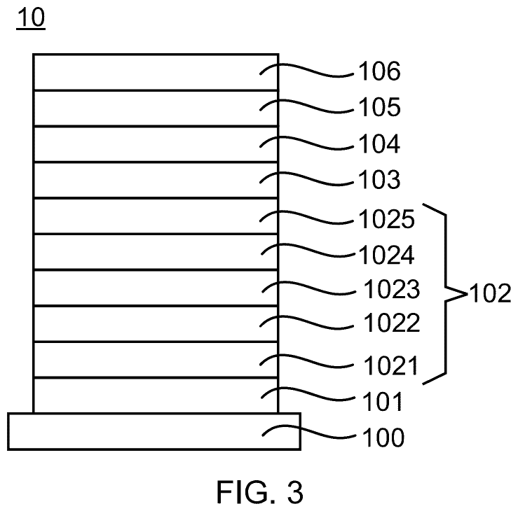

With reference to FIG. 2, embodiments of the present disclosure provide an OLED 10. The OLED 10 includes a substrate 100, an anode 101, a cathode 103, an organic light-emitting functional layer 102 and a cover layer 104. The organic light-emitting functional layer 102 may include a light-emitting layer 1023, a hole transport layer 1022, a hole injection layer 1021, an electron transport layer 1024, an electron injection layer 1025, etc., or may include only the light-emitting layer 1023 and one or more of the other layers. The hole transport layer 1022 contains one or more of compounds as shown in general formula (I) above. In some examples, with reference to FIG. 3, a protective layer 105 and/or an encapsulation layer 106 are further provided on the cover layer 104.

The substrate in embodiments of the present disclosure may be any substrate selected from substrates applied in typical organic light-emitting apparatuses. The substrate may be a glass substrate or a transparent plastic substrate, or may be a substrate of an opaque material such as silicon or stainless steel, or may be a flexible polyimide (PI) film. Different substrates are different in mechanical strength, thermal stability, transparency, surface smoothness and waterproofness; and depending on natures of the different substrates, the different substrates are used in different directions.

Materials of the hole injection layer, the electron injection layer and the light-emitting layer may use any materials selected from known relevant materials for OLED devices.

As host and guest materials capable of generating blue fluorescence, green fluorescence and blue-green fluorescence, the materials need to have not only an extremely high fluorescence quantum light-emitting efficiency but also an appropriate energy level.

The present disclosure is specifically described by means of specific embodiments below. All raw materials and solvents in synthetic embodiments are purchased commercially unless otherwise specified. The solvents are all used directly without further treatment.

### EMBODIMENTS

### Embodiment 1: Synthesis of Compound H001

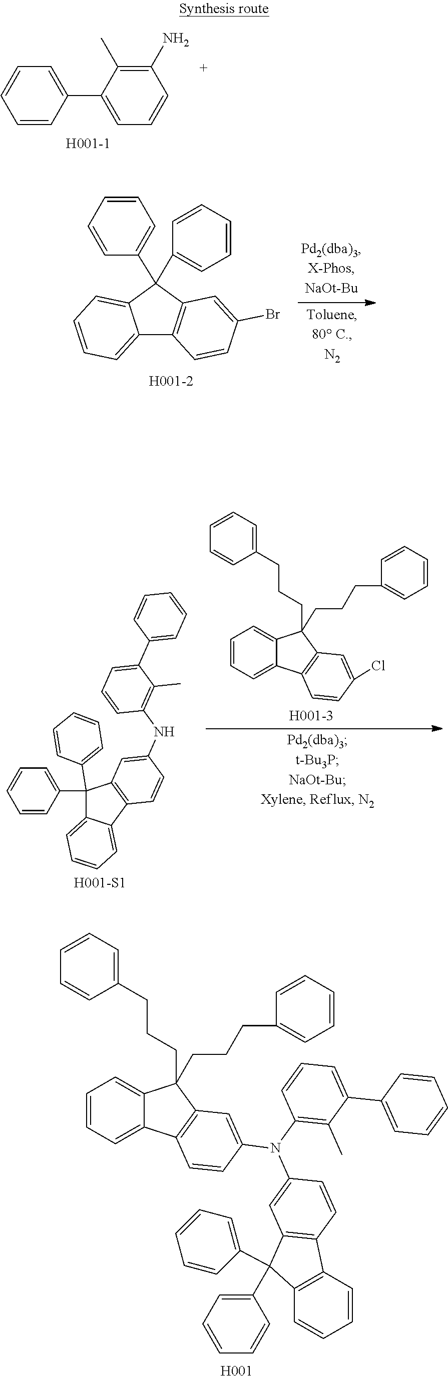

### Synthesis Methods:

1) Compounds H001-1 (18.3 g) and H001-2 (27.3 g), Pd₂(dba)₃ (1.2 g), 2-dicyclohexylphosphino-2′,4′,6′-triisopropylbiphenyl (X-Phos) (1.5 g) and sodium tert-butoxide (NaOt-Bu) (10 g) are sequentially added to 500 mL of toluene, air in such a reaction system is fully substituted with nitrogen under stirring at room temperature for five times, and then a reaction is carried out at 80° C. for 8 hours; and after the reaction is completed, the temperature is reduced to room temperature, water washing is performed for liquid separation so as to obtain an organic phase, and the organic phase is concentrated, and then separated by means of column chromatography, such that an intermediate product H001-S1 (30 g) is obtained.
2) The intermediate product H001-S1 (30 g), compound H001-3 (48 g), Pd₂ (dba)₃ (1 g), X-Phos (1.3 g), and sodium tert-butoxide (9 g) are sequentially added to 400 ml of xylene, air in such a reaction system is fully substituted with nitrogen under stirring at room temperature for five times, and then a reflux reaction is carried out for 12 hours; and after the reaction is completed, the temperature is reduced to room temperature, water washing is performed for liquid separation so as to obtain an organic phase, and the organic phase is concentrated, and then separated by means of column chromatography, such that a target product H001 (55 g) is obtained. Through liquid chromatography-mass spectrometry (LC-MS) analysis performed on the target product, a following result of LC-MS (m/z) (M+) is obtained: a theoretical value being 899.45, and a tested value being 900.15.

### Embodiment 2: Synthesis of Compound H005

With reference to the synthesis steps and reaction conditions of Embodiment 1, compound H005 is synthesized. Through LC-MS analysis, a following result of LC-MS (m/z) (M+) is obtained: a theoretical value being 699.39, and a tested value being 699.95.

### Embodiment 3: Synthesis of Compound H016

With reference to the synthesis steps and reaction conditions of Embodiment 1, compound H016 is synthesized. Through LC-MS analysis, a following result of LC-MS (m/z) (M+) is obtained: a theoretical value being 985.43, and a tested value being 986.17.

### Embodiment 4: Synthesis of Compound H027

With reference to the synthesis steps and reaction conditions of Embodiment 1, compound H027 is synthesized. Through LC-MS analysis, a following result of LC-MS (m/z) (M+) is obtained: a theoretical value being 951.48, and a tested value being 952.08.

### Embodiment 5: Synthesis of Compound H038

With reference to the synthesis steps and reaction conditions of Embodiment 1, compound H038 is synthesized. Through LC-MS analysis, a following result of LC-MS (m/z) (M+) is obtained: a theoretical value being 847.42, and a tested value being 848.06.

### Embodiment 6: Synthesis of Compound H047

With reference to the synthesis steps and reaction conditions of Embodiment 1, compound H047 is synthesized. Through LC-MS analysis, a following result of LC-MS (m/z) (M+) is obtained: a theoretical value being 964.48, and a tested value being 965.10.

### Embodiment 7: Synthesis of Compound H052

With reference to the synthesis steps and reaction conditions of Embodiment 1, compound H052 is synthesized. Through LC-MS analysis, a following result of LC-MS (m/z) (M+) is obtained: a theoretical value being 901.43, and a tested value being 902.09.

### Embodiment 8: Synthesis of Compound H057

With reference to the synthesis steps and reaction conditions of Embodiment 1, compound H057 is synthesized. Through LC-MS analysis, a following result of LC-MS (m/z) (M+) is obtained: a theoretical value being 976.48, and a tested value being 977.12.

### Embodiment 9: Synthesis of Compound H058

With reference to the synthesis steps and reaction conditions of Embodiment 1, compound H058 is synthesized. Through LC-MS analysis, a following result of LC-MS (m/z) (M+) is obtained: a theoretical value being 915.44, and a tested value being 916.12.

### Embodiment 10: Synthesis of Compound H065

With reference to the synthesis steps and reaction conditions of Embodiment 1, compound H065 is synthesized. Through LC-MS analysis, a following result of LC-MS (m/z) (M+) is obtained: a theoretical value being 851.45, and a tested value being 852.11.

### Embodiment 11: Synthesis of Compound H068

With reference to the synthesis steps and reaction conditions of Embodiment 1, compound H068 is synthesized. Through LC-MS analysis, a following result of LC-MS (m/z) (M+) is obtained: a theoretical value being 912.54, and a tested value being 913.26.

### Embodiment 12: Synthesis of Compound H070

With reference to the synthesis steps and reaction conditions of Embodiment 1, compound H070 is synthesized. Through LC-MS analysis, a following result of LC-MS (m/z) (M+) is obtained: a theoretical value being 845.41, and a tested value being 846.11.

### Embodiment 13: Synthesis of Compound H080

With reference to the synthesis steps and reaction conditions of Embodiment 1, compound H080 is synthesized. Through LC-MS analysis, a following result of LC-MS (m/z) (M+) is obtained: a theoretical value being 793.46, and a tested value being 794.10.

### Embodiment 14: Synthesis of Compound H085

With reference to the synthesis steps and reaction conditions of Embodiment 1, compound H085 is synthesized. Through LC-MS analysis, a following result of LC-MS (m/z) (M+) is obtained: a theoretical value being 687.39, and a tested value being 687.95.

### Embodiment 15: Synthesis of Compound H086

With reference to the synthesis steps and reaction conditions of Embodiment 1, compound H086 is synthesized. Through LC-MS analysis, a following result of LC-MS (m/z) (M+) is obtained: a theoretical value being 735.39, and a tested value being 736.07.

### Embodiment 16: Synthesis of Compound H089

With reference to the synthesis steps and reaction conditions of Embodiment 1, compound H089 is synthesized. Through LC-MS analysis, a following result of LC-MS (m/z) (M+) is obtained: a theoretical value being 925.46, and a tested value being 926.20.

### Embodiment 17: Synthesis of Compound H091

With reference to the synthesis steps and reaction conditions of Embodiment 1, compound H091 is synthesized. Through LC-MS analysis, a following result of LC-MS (m/z) (M+) is obtained: a theoretical value being 801.43, and a tested value being 802.05.

### Embodiment 18: Synthesis of Compound H095

With reference to the synthesis steps and reaction conditions of Embodiment 1, compound H095 is synthesized. Through LC-MS analysis, a following result of LC-MS (m/z) (M+) is obtained: a theoretical value being 739.42, and a tested value being 740.04.

Application examples, in which several of the hole transport materials described in the embodiments of the present disclosure are applied in OLED devices, are listed below, so as to further illustrate the beneficial effects of the compounds in the embodiments of the present disclosure. Materials used in the examples are purchased commercially or synthesized in-house.

### Fabrication of OLED Devices

A reference fabrication method for the device embodiments of the present disclosure is as follows: evaporating 50-500 nm of indium tin oxide/Ag/indium tin oxide (ITO/Ag/ITO) on an alkali-free glass substrate to serve as an anode; sequentially evaporating a hole injection layer (5-20 nm), a hole transport layer (50-150 nm), a light-emitting auxiliary layer (5-120 nm), a light-emitting layer (20-50 nm), a hole blocking layer (5-20 nm), an electron transport layer (20-80 nm) and an electron injection layer (0.5-10 nm) on the anode in a stacked manner; then, co-evaporating Mg and Ag (a weight ratio of 1:9, 10-50 nm) to form a semi-transparent cathode; then, evaporating a cover layer compound; and finally, encapsulating such a light-emitting device by using an epoxy resin adhesive under nitrogen atmosphere.

In an embodiment, a fabrication method of an OLED device provided in embodiments of the present disclosure is as follows. An alkali-free glass substrate is first washed with isopropanol by using an ultrasonic cleaning instrument for 15 minutes, and then ultraviolet (UV) ozone washing treatment is performed in air for 30 minutes; on the treated substrate, 100 nm of ITO/Ag/ITO is evaporated by using a vacuum evaporation method to form an anode; on the anode, a hole injection layer (containing compound HT and compound PD, a weight ratio of compound HT to compound PD being 97:3, a total thickness of the hole injection layer being 10 nm), a hole transport layer (containing compound H001, a thickness being 135 nm), a light-emitting auxiliary layer (containing compound BP, a thickness being 5 nm), a blue light-emitting layer (respectively using compound BH and compound BD as a host material and a dopant material, a weight ratio of the host material to the dopant material, i.e., a weight ratio of compound BH to compound BD, being 98:2, a total thickness of the blue light-emitting layer being 20 nm), a hole blocking layer (containing compound HBL, a thickness being 5 nm), an electron transport layer (containing compound ET and compound Liq, a weight ratio of compound ET to compound Liq being 1:1, a total thickness of the electron transport layer being 30 nm) and an electron injection layer (containing ytterbium (Yb), a thickness being 10 nm) are sequentially evaporated on the substrate in a stacked manner; Mg and Ag (a weight ratio of 1:9, a thickness being 14 nm) are co-evaporated to form a semi-transparent cathode; then, compound CPL (70 nm) is evaporated to form a cover layer. This embodiment is recorded as Application Example 1. Molecular structure formulas of the relevant materials are as shown below (the structures below being only examples, which does not mean that embodiments of the present disclosure are limited thereto):

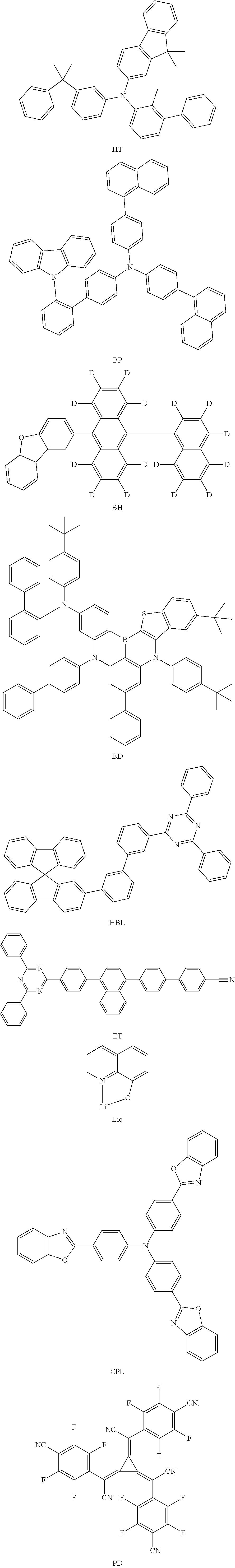

OLED devices in Application Examples 2 to 18 and Comparison Example 1 are each fabricated with reference to the method provided in Application Example 1. The only difference lies in that, Application Examples 2 to 18 and Comparison Example 1 respectively use corresponding compounds as listed in Table 1, instead of compound H001 in Application Example 1, to serve as hole transport materials. A structure of compound Ref-1 used in Comparison Example 1 is as follows:

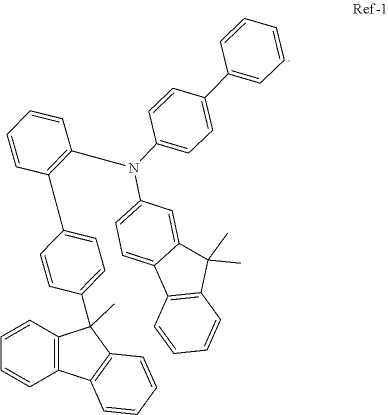 [SMILES: CC1(C)c2ccccc2-c2ccc(N(c3ccc(-c4ccccc4)cc3)c3ccccc3-c3ccc(C4(C)c5ccccc5-c5ccccc54)cc3)cc21]

### Performance Evaluation of OLED Devices

Currents of each OLED device at different voltages are tested by means of Keithley 2365A digital nanovoltmeter, and then current densities of the OLED device at the different voltages are obtained by dividing the currents by a light-emitting area of the OLED device; brightnesses and radiation energy flux densities of the OLED device at the different voltages are tested by means of Konicaminolta CS-2000 spectroradiometer; and according to the current densities and the brightnesses of the OLED device at the different voltages, an operation voltage Volt and a current efficiency (cd/A) at the same current density (10 mA/cm2) are obtained; BI=E/CIEy, where BI refers to a blue index of blue light, which is also a parameter for measuring a light-emitting efficiency of the blue light, E refers to the current efficiency, and CIEy refers to a color point of a vertical coordinate obtained by substituting a full band spectrum of the device into the software CIE1930 for integration. Test data are shown in Table 1.

TABLE 1
Devices of hole transport material application examples
and electroluminescent properties thereof

| Device | Hole Transport | Volt | BI | Lh95 |
|---|---|---|---|---|
| Embodiment | Material | (V) | (cd/A/CIEy) | (hr) |
| Application | H001 | 3.10 | 228.01 | 104 |
| Example 1 | | | | |
| Application | H005 | 3.12 | 227.78 | 105 |
| Example 2 | | | | |
| Application | H016 | 3.16 | 228.08 | 102 |
| Example 3 | | | | |
| Application | H027 | 3.11 | 225.86 | 103 |
| Example 4 | | | | |
| Application | H038 | 3.09 | 226.67 | 101 |
| Example 5 | | | | |
| Application | H047 | 3.12 | 228.30 | 100 |
| Example 6 | | | | |
| Application | H052 | 3.16 | 227.06 | 98 |
| Example 7 | | | | |
| Application | H057 | 3.08 | 226.05 | 104 |
| Example 8 | | | | |
| Application | H058 | 3.12 | 227.78 | 97 |
| Example 9 | | | | |
| Application | H065 | 3.10 | 230.00 | 99 |
| Example 10 | | | | |
| Application | H068 | 3.11 | 229.01 | 96 |
| Example 11 | | | | |
| Application | H070 | 3.09 | 226.35 | 98 |
| Example 12 | | | | |
| Application | H080 | 3.15 | 225.38 | 95 |
| Example 13 | | | | |
| Application | H085 | 3.12 | 226.85 | 93 |
| Example 14 | | | | |
| Application | H086 | 3.14 | 228.39 | 99 |
| Example 15 | | | | |
| Application | H089 | 3.05 | 227.34 | 94 |
| Example 16 | | | | |
| Application | H091 | 3.23 | 226.61 | 96 |
| Example 17 | | | | |
| Application | H095 | 3.10 | 228.01 | 93 |
| Example 18 | | | | |
| Comparison | Ref-1 | 3.41 | 205.33 | 78 |
| Example 1 | | | | |

As can be seen from Table 1, compared with the OLED device in Comparison Example 1, the OLED devices in Application Examples 1 to 18 have lower operating voltages and higher BI light-emitting efficiencies. The improved performances of the application examples are based on the fact that the compound materials of the embodiments of the present disclosure have better film-forming stabilities and transport properties. The hole transport material in the embodiments of the present disclosure uses triarylamine as a parent nucleus, and uses an aryl-substituted, alkyl-substituted or aryl and alkyl co-substituted fluorenyl group, an aryl-substituted or alkyl-substituted fluorenyl group or a heteroaryl group, and a ortho-substituted aryl group as three side chains to combine with each other, which enhances a rigid conjugation effect of the compound. Therefore, the compound has a good thermally stability, and a significant effect on light-emitting properties of the compound is achieved, thereby improving a light-emitting efficiency of the device, well achieving electron and hole transport equalization and an exciton conversion rate, and reducing a power consumption of the device.

These specific embodiments are only explanations of the present disclosure, but not limitations of the present disclosure. Those skilled in the art may make modifications, without creative contribution, to these embodiments according to needs after reading this description, but those modifications shall be included in the scope, which are protected by the patent law, of the claims of the present disclosure.

What is claimed is:

1. A hole transport material, containing a compound represented by formula I-1: 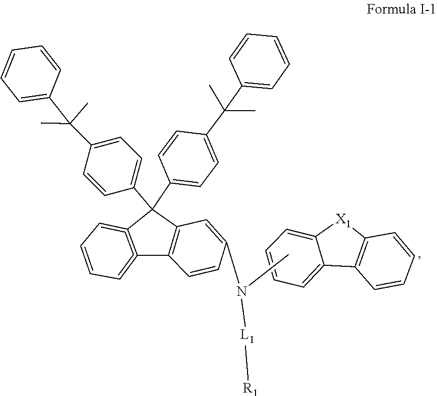
wherein X₁ independently represents CRₐR_{b}, NR_{c}, an O atom or an S atom; and Rₐ, R_{b} and R_{c} are each independently selected from a C1-C24 alkyl group, a C6-C30 aryl group and a C5-C30 heteroaryl group; and
L₁ is selected from a single bond and a substituted or unsubstituted C6-C30 arylene group, the substituted C6-C30 arylene group having mono-substitution, di-substitution or a maximum permissible number of substitutions, and in a case of a substituent is involved, the substituent being hydrogen, deuterium, a C1-C24 alkyl group or a monocyclic, annelated, bridged cyclic, spirocyclic or fused cyclic C3-C20 cycloalkyl group; and R₁ is selected from a C1-C24 alkyl-substituted, or monocyclic, annelated, bridged cyclic, spirocyclic or fused cyclic C3-C20 cycloalkyl-substituted or unsubstituted C6-C30 aryl group, and a C6-C30 aryl-substituted or unsubstituted C4-C36 heteroaryl group.

2. The hole transport material according to claim 1, wherein X₁ represents CRₐR_{b}, and Rₐ and R_{b} are each independently selected from a methyl group and a phenyl group.

3. The hole transport material according to claim 1, wherein X₁ represents NR_{c}, and R_{c} is a phenyl group.

4. The hole transport material according to claim 1, wherein L₁ is selected from a single bond and a C1-C10 alkyl-substituted C6-C30 arylenen group; and R₁ is independently selected from a C1-C5 alkyl-substituted, C3-C10 cycloalkyl-substituted or unsubstituted C6-C30 aryl group, and a C6-C30 aryl-substituted or unsubstituted C4-C36 heteroaryl group.

5. The hole transport material according to claim 1, wherein L₁ is independently selected from a single bond, a phenylene group, a biphenylene group, a methylphenylene group, a dimethylphenylene group and an adamantyl-substituted phenylene group; and R₁ is independently selected from a phenyl group, a methylphenyl group, a dimethylphenyl group, a phenylcarbazolyl group and a dibenzofuranyl group.

6. The hole transport material according to claim 1, wherein the hole transport material has any chemical structure selected from chemical structures shown below:

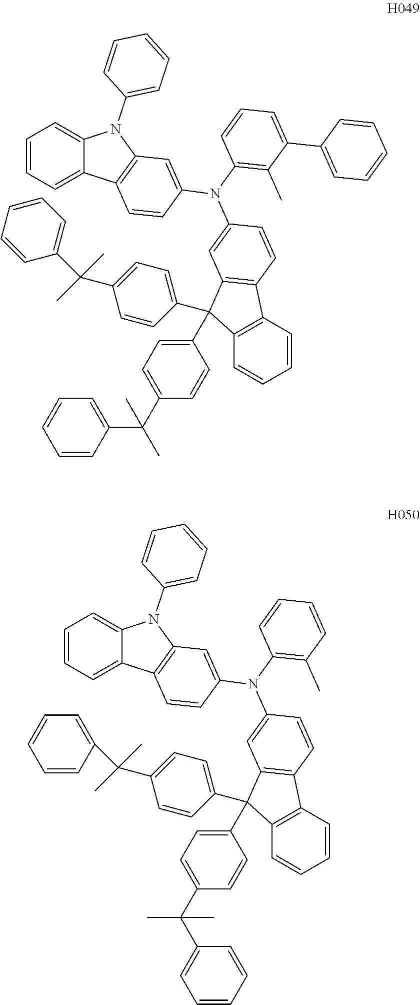 [SMILES: Cc1c(-c2ccccc2)cccc1N(c1ccc2c(c1)C(c1ccc(C(C)(C)c3ccccc3)cc1)(c1ccc(C(C)(C)c3ccccc3)cc1)c1ccccc1-2)c1ccc2c3ccccc3n(-c3ccccc3)c2c1 ; Cc1ccccc1N(c1ccc2c(c1)C(c1ccc(C(C)(C)c3ccccc3)cc1)(c1ccc(C(C)(C)c3ccccc3)cc1)c1ccccc1-2)c1ccc2c3ccccc3n(-c3ccccc3)c2c1]

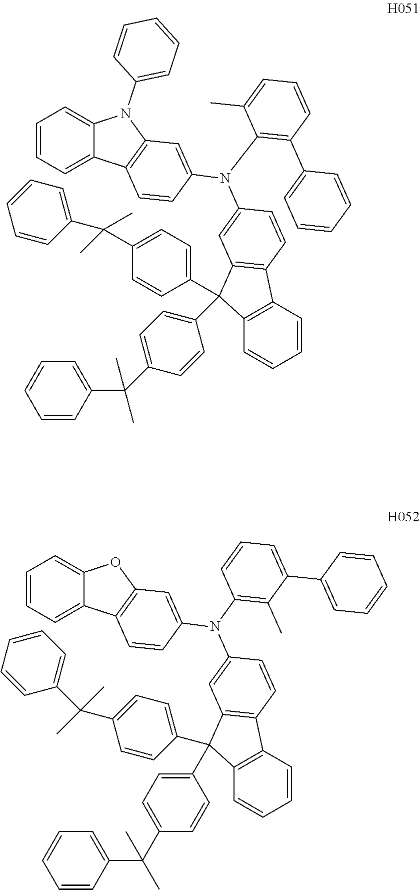 [SMILES: Cc1cccc(-c2ccccc2)c1N(c1ccc2c(c1)C(c1ccc(C(C)(C)c3ccccc3)cc1)(c1ccc(C(C)(C)c3ccccc3)cc1)c1ccccc1-2)c1ccc2c3ccccc3n(-c3ccccc3)c2c1 ; Cc1c(-c2ccccc2)cccc1N(c1ccc2c(c1)C(c1ccc(C(C)(C)c3ccccc3)cc1)(c1ccc(C(C)(C)c3ccccc3)cc1)c1ccccc1-2)c1ccc2c(c1)oc1ccccc12]

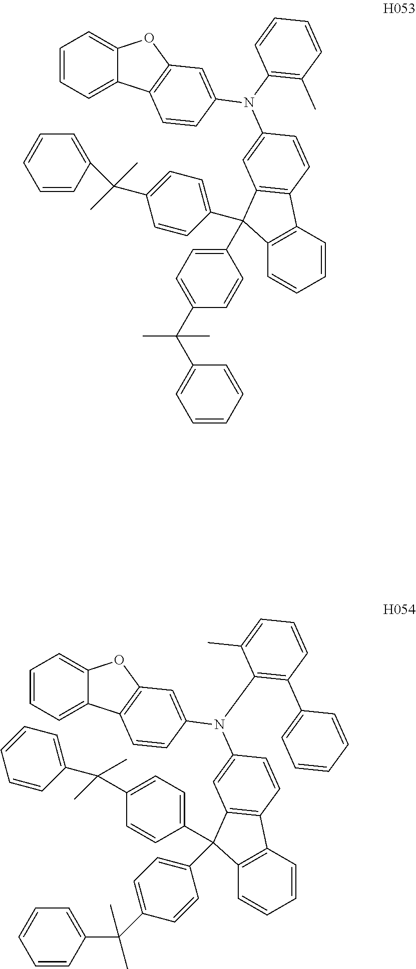 [SMILES: Cc1ccccc1N(c1ccc2c(c1)C(c1ccc(C(C)(C)c3ccccc3)cc1)(c1ccc(C(C)(C)c3ccccc3)cc1)c1ccccc1-2)c1ccc2c(c1)oc1ccccc12 ; Cc1cccc(-c2ccccc2)c1N(c1ccc2c(c1)C(c1ccc(C(C)(C)c3ccccc3)cc1)(c1ccc(C(C)(C)c3ccccc3)cc1)c1ccccc1-2)c1ccc2c(c1)oc1ccccc12]

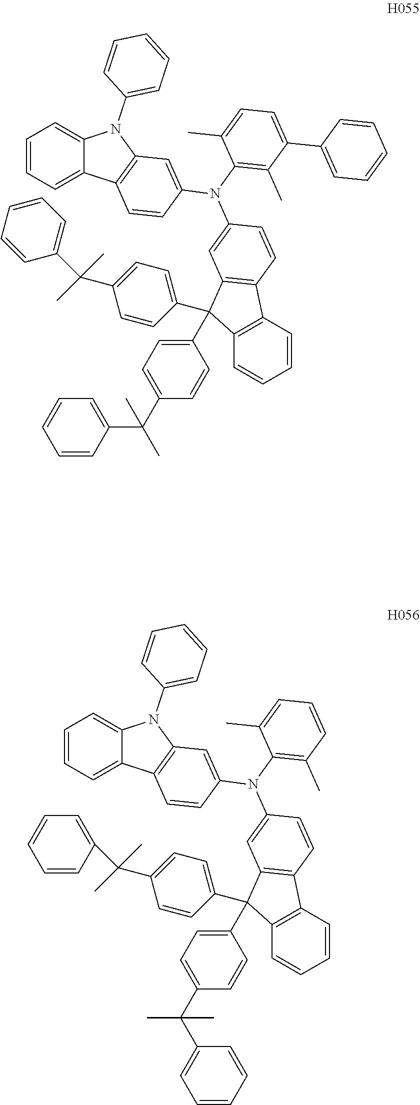 [SMILES: Cc1ccc(-c2ccccc2)c(C)c1N(c1ccc2c(c1)C(c1ccc(C(C)(C)c3ccccc3)cc1)(c1ccc(C(C)(C)c3ccccc3)cc1)c1ccccc1-2)c1ccc2c3ccccc3n(-c3ccccc3)c2c1 ; Cc1cccc(C)c1N(c1ccc2c(c1)C(c1ccc(C(C)(C)c3ccccc3)cc1)(c1ccc(C(C)(C)c3ccccc3)cc1)c1ccccc1-2)c1ccc2c3ccccc3n(-c3ccccc3)c2c1]

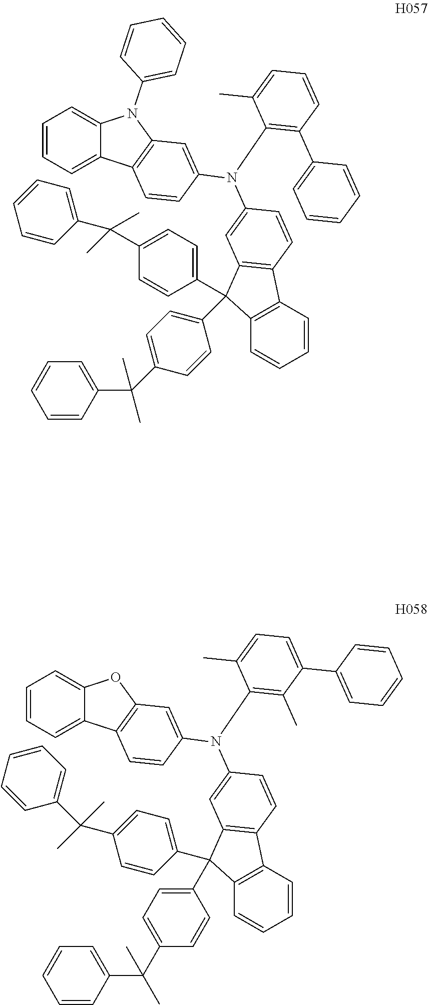 [SMILES: Cc1cccc(-c2ccccc2)c1N(c1ccc2c(c1)C(c1ccc(C(C)(C)c3ccccc3)cc1)(c1ccc(C(C)(C)c3ccccc3)cc1)c1ccccc1-2)c1ccc2c3ccccc3n(-c3ccccc3)c2c1 ; Cc1ccc(-c2ccccc2)c(C)c1N(c1ccc2c(c1)C(c1ccc(C(C)(C)c3ccccc3)cc1)(c1ccc(C(C)(C)c3ccccc3)cc1)c1ccccc1-2)c1ccc2c(c1)oc1ccccc12]

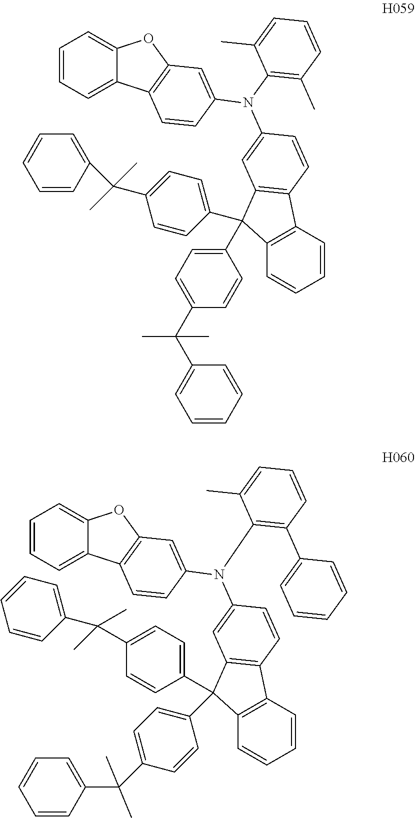 [SMILES: Cc1cccc(C)c1N(c1ccc2c(c1)C(c1ccc(C(C)(C)c3ccccc3)cc1)(c1ccc(C(C)(C)c3ccccc3)cc1)c1ccccc1-2)c1ccc2c(c1)oc1ccccc12 ; Cc1cccc(-c2ccccc2)c1N(c1ccc2c(c1)C(c1ccc(C(C)(C)c3ccccc3)cc1)(c1ccc(C(C)(C)c3ccccc3)cc1)c1ccccc1-2)c1ccc2c(c1)oc1ccccc12]

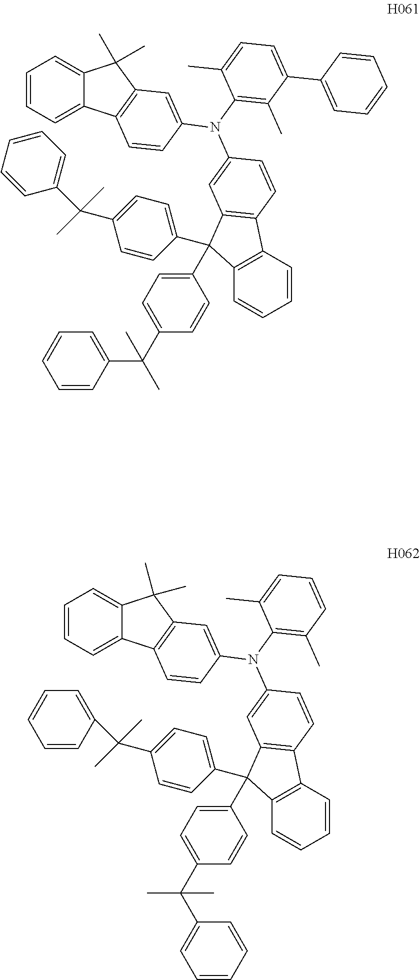 [SMILES: Cc1ccc(-c2ccccc2)c(C)c1N(c1ccc2c(c1)C(C)(C)c1ccccc1-2)c1ccc2c(c1)C(c1ccc(C(C)(C)c3ccccc3)cc1)(c1ccc(C(C)(C)c3ccccc3)cc1)c1ccccc1-2 ; Cc1cccc(C)c1N(c1ccc2c(c1)C(C)(C)c1ccccc1-2)c1ccc2c(c1)C(c1ccc(C(C)(C)c3ccccc3)cc1)(c1ccc(C(C)(C)c3ccccc3)cc1)c1ccccc1-2]

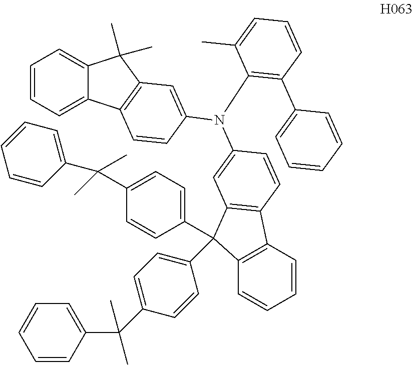 [SMILES: Cc1cccc(-c2ccccc2)c1N(c1ccc2c(c1)C(C)(C)c1ccccc1-2)c1ccc2c(c1)C(c1ccc(C(C)(C)c3ccccc3)cc1)(c1ccc(C(C)(C)c3ccccc3)cc1)c1ccccc1-2]

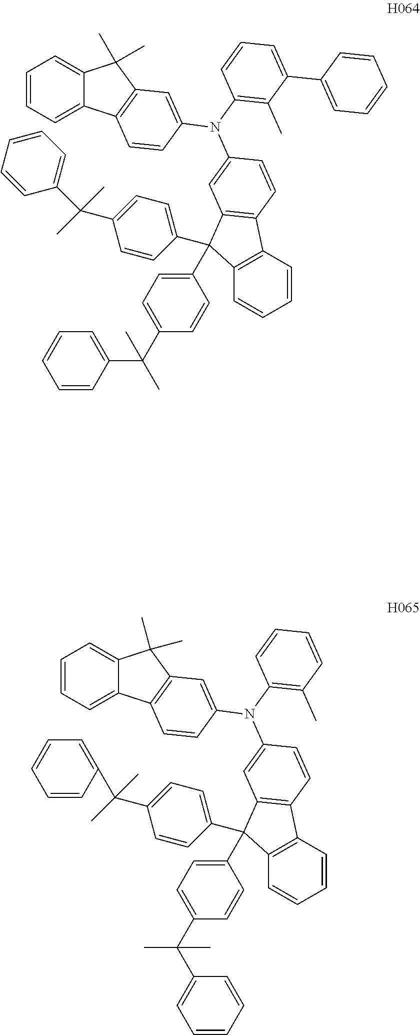 [SMILES: Cc1c(-c2ccccc2)cccc1N(c1ccc2c(c1)C(C)(C)c1ccccc1-2)c1ccc2c(c1)C(c1ccc(C(C)(C)c3ccccc3)cc1)(c1ccc(C(C)(C)c3ccccc3)cc1)c1ccccc1-2 ; Cc1ccccc1N(c1ccc2c(c1)C(C)(C)c1ccccc1-2)c1ccc2c(c1)C(c1ccc(C(C)(C)c3ccccc3)cc1)(c1ccc(C(C)(C)c3ccccc3)cc1)c1ccccc1-2]

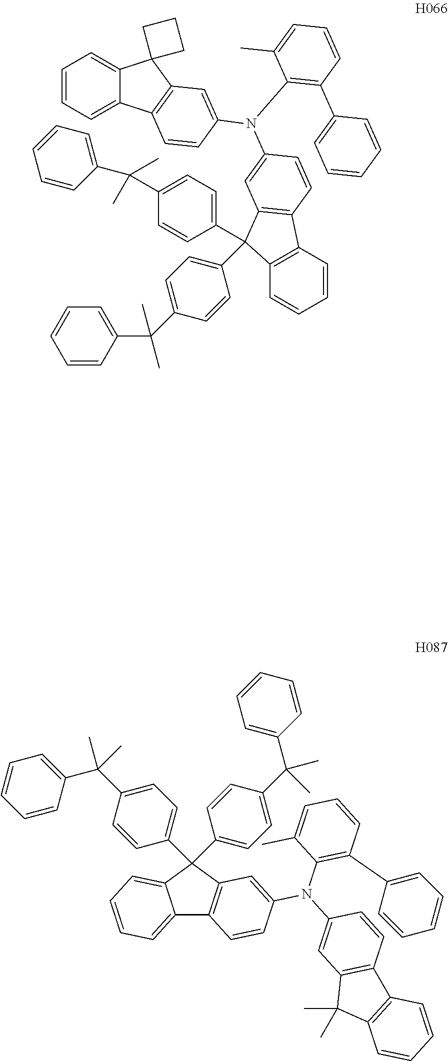 [SMILES: Cc1cccc(-c2ccccc2)c1N(c1ccc2c(c1)C1(CCC1)c1ccccc1-2)c1ccc2c(c1)C(c1ccc(C(C)(C)c3ccccc3)cc1)(c1ccc(C(C)(C)c3ccccc3)cc1)c1ccccc1-2 ; Cc1cccc(-c2ccccc2)c1N(c1ccc2c(c1)C(C)(C)c1ccccc1-2)c1ccc2c(c1)C(c1ccc(C(C)(C)c3ccccc3)cc1)(c1ccc(C(C)(C)c3ccccc3)cc1)c1ccccc1-2]

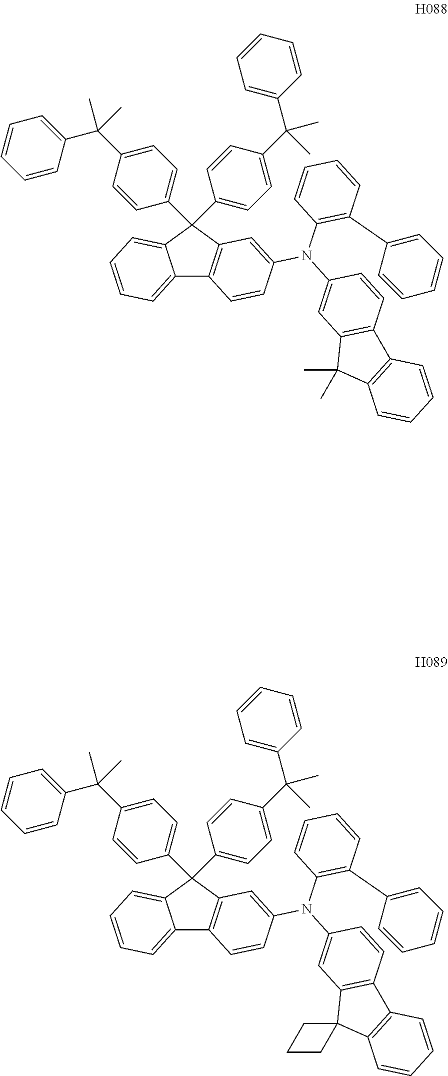 [SMILES: CC(C)(c1ccccc1)c1ccc(C2(c3ccc(C(C)(C)c4ccccc4)cc3)c3ccccc3-c3ccc(N(c4ccc5c(c4)C(C)(C)c4ccccc4-5)c4ccccc4-c4ccccc4)cc32)cc1 ; CC(C)(c1ccccc1)c1ccc(C2(c3ccc(C(C)(C)c4ccccc4)cc3)c3ccccc3-c3ccc(N(c4ccc5c(c4)C4(CCC4)c4ccccc4-5)c4ccccc4-c4ccccc4)cc32)cc1]

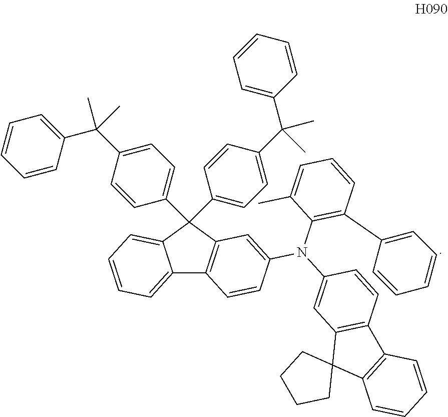 [SMILES: Cc1cccc(-c2ccccc2)c1N(c1ccc2c(c1)C1(CCCC1)c1ccccc1-2)c1ccc2c(c1)C(c1ccc(C(C)(C)c3ccccc3)cc1)(c1ccc(C(C)(C)c3ccccc3)cc1)c1ccccc1-2]

7. An organic electroluminescent device, comprising:
a substrate;
a first electrode, which is disposed on the substrate;
an organic light-emitting functional layer, which is disposed on the first electrode; and
a second electrode, which is disposed on the organic light-emitting functional layer, wherein
the organic light-emitting functional layer comprises a hole transport layer, and the hole transport layer comprises the hole transport material according to claim 1.

8. An organic electroluminescent device, comprising:
a substrate;
a first electrode, which is disposed on the substrate;
an organic light-emitting functional layer, which is disposed on the first electrode; and
a second electrode, which is disposed on the organic light-emitting functional layer, wherein
the organic light-emitting functional layer comprises a hole transport layer, and the hole transport layer comprises the hole transport material according to claim 2.

9. An organic electroluminescent device, comprising:
a substrate;
a first electrode, which is disposed on the substrate;
an organic light-emitting functional layer, which is disposed on the first electrode; and
a second electrode, which is disposed on the organic light-emitting functional layer, wherein
the organic light-emitting functional layer comprises a hole transport layer, and the hole transport layer comprises the hole transport material according to claim 3.

10. An organic electroluminescent device, comprising:
a substrate;
a first electrode, which is disposed on the substrate;
an organic light-emitting functional layer, which is disposed on the first electrode; and
a second electrode, which is disposed on the organic light-emitting functional layer, wherein
the organic light-emitting functional layer comprises a hole transport layer, and the hole transport layer comprises the hole transport material according to claim 4.

11. An organic electroluminescent device, comprising:
a substrate;
a first electrode, which is disposed on the substrate;
an organic light-emitting functional layer, which is disposed on the first electrode; and
a second electrode, which is disposed on the organic light-emitting functional layer, wherein
the organic light-emitting functional layer comprises a hole transport layer, and the hole transport layer comprises the hole transport material according to claim 5.

12. A formulation, containing the hole transport material according to claim 1 and at least one solvent.

13. A formulation, containing the hole transport material according to claim 2 and at least one solvent.

14. A formulation, containing the hole transport material according to claim 3 and at least one solvent.

15. A formulation, containing the hole transport material according to claim 4 and at least one solvent.

16. A formulation, containing the hole transport material according to claim 5 and at least one solvent.

17. A display apparatus, comprising the organic electroluminescent device according to claim 7.

18. A lighting apparatus, comprising the organic electroluminescent device according to claim 7.
